(19) United States
(12) Patent Application Publication
(10) Pub. No.: US 2007/0185093 A1
(43) Pub. Date: Aug. 9, 2007

(54) Benzotriazepine derivatives and their use as gastrin and cholecystokinin receptor ligands

(76) Inventors: Ildiko Maria Buck, London (GB); Iain Mair McDonald, London (GB)

Correspondence Address: PROSKAUER ROSE LLP, 1001 PENNSYLVANIA AVE, N.W.,, SUITE 400 SOUTH, WASHINGTON, DC, 20004, US

(21) Appl. No.: 10/556,585

(22) PCT Filed: May 12, 2004
(86) PCT No.: PCT/GB04/02027

§ 371 Date: Nov. 30, 2006

(30) Foreign Application Priority Data
May 12, 2003 (GB) 0310865.1

(51) Int. Cl. A61K 31/5513; C07D 255/04

(52) U.S. Cl. 514/221; 540/501

(57) ABSTRACT

This invention relates to a compound of formula (I). The compound is useful for the treatment of gastrin related disorders.

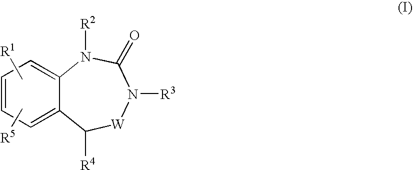

This application claims priority from U.K. patent application No. 0310865.1, the entire contents of which is hereby incorporated by reference.

This invention relates to gastrin and cholecystokinin (CCK) receptor ligands. (The receptor previously known as the CCK_{B}/gastrin receptor is now termed the CCK₂ receptor). The invention also relates to methods for preparing such ligands and to compounds which are useful intermediates in such methods. The invention further relates to pharmaceutical compositions comprising such ligands and methods for preparing such pharmaceutical compositions.

Gastrin and the cholecystokinins are structurally related neuropeptides which exist in gastrointestinal tissue and the central nervous system (Mutt V., Gastrointestinal Hormones, Glass G. B. J., ed., Raven Press, New York, p. 169; Nisson G., ibid., p. 127).

Gastrin is one of the three primary stimulants of gastric acid secretion. Several forms of gastrin are found including 34-, 17- and 14-amino acid species with the minimum active fragment being the C-terminal tetrapeptide (TrpMetAspPhe-NH₂) which is reported in the literature to have full pharmacological activity (Tracy H. J. and Gregory R. A., Nature (London), 1964, 204, 935). Much effort has been devoted to the synthesis of analogues of this tetrapeptide (and the N-protected derivative Boc-TrpMetAspPhe-NH₂) in an attempt to elucidate the relationship between structure and activity.

Natural cholecystokinin is a 33 amino acid peptide (CCK-33), the C-terminal 5 amino acids of which are identical to those of gastrin. Also found naturally is the C-terminal octapeptide (CCK-8) of CCK-33.

The cholecystokinins are reported to be important in the regulation of appetite. They stimulate intestinal mobility, gall bladder contraction, pancreatic enzyme secretion and are known to have a trophic action on the pancreas. They also inhibit gastric emptying and have various effects in the central nervous system.

Compounds which bind to cholecystokinin and/or gastrin receptors are important because of their potential pharmaceutical use as antagonists, inverse agonists or partial agonists of the natural peptides. Such compounds are described herein as ligands. The term ligand as used herein means either an antagonist, partial or full agonist, or an inverse agonist. Usually, the term ligand refers to an antagonist.

A number of gastrin ligands have been proposed for various therapeutic applications, including the prevention of gastrin-related disorders including gastrointestinal ulcers, dyspepsia, reflux oesophagitis (gastroesophageal reflux disease (GERD), both erosive and non-erosive) by reduction in gastric acid secretion and/or improving impaired motor activity at the lower oesophageal sphincter, Zollinger-Ellison syndrome, Barrett's oesophagus (specialized intestinal metaplasia of distal oesophagus), ECL cell hyperplasia, rebound hypersecretion (following cessation of anti-secretory therapy), ECL-derived gastric polyps most commonly found in patients with atrophic gastritis both with (pernicious anaemia) or without vitamin B12 deficiency, antral G cell hyperplasia and other conditions in which lower gastrin activity or lower acid secretion is desirable. The hormone has also been shown to have a trophic action on cells and so an antagonist may be expected to be useful in the treatment of cancers, particularly in the GI tract, more particularly in the stomach, oesophagus and colo-rectal areas. Tumours found in other organs such as the pancreas, lung (small cell lung carcinomas) and thyroid (thyroid medullary tumours) may also be treated.

Other possible uses are in the potentiation of opiate (for example morphine) analgesia. Moreover, ligands for cholecystokinin receptors in the brain (so-called CCK₂ receptors) have been claimed to possess anxiolytic activity.

A known antagonist of the CCK₂ receptor is L-365,260 (M. G. Bock et al., J. Med Chem., 1989, 32, 13-16), which is based on a benzodiazepine structure. In the rat stomach assay described hereinbelow, L-365,260 was shown to have an affinity of pK_{B}=7.61±0.12 for the CCK₂ receptor (S. B. Kalindjian et al., J. Med. Chem., 1994, 37, 3671-3).

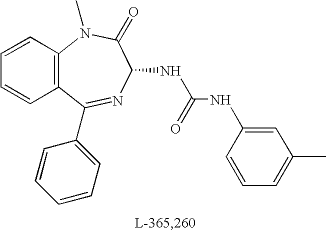

More recently, another benzodiazepine, YF476, was developed as a potent CCK₂ antagonist (A. Nishida et al., Journal of Pharmacology and Experimental Therapeutics, 1994, 269, 725-731). In rat cortical membranes, YF476 was found to have an affinity pKᵢ of 10.17±0.03 for the CCK₂ receptor.

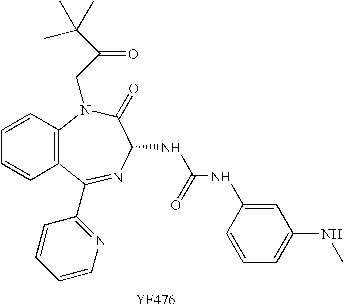

L-365,260 and YF476 are structurally closely related. Both compounds are dihydro-1,4-benzodiazepines. Reduction of the imine group within the 7-membered ring to produce tetrahydro-1,4-benzodiazepines has only ever been reported in either the CCK or gastrin area by Evans et al. (J. Med. Chem., (1987), 30(7), 1229-1239), who worked on a series of dihydro-1,4-benzodiazepines that were predominantly CCK₁ antagonists. This reduction was found to be generally detrimental to activity.

U.S. Pat. No. 5,091,381 describes dihydro-1,3,4-benzotriazepines, which are said to bind to peripheral benzodiazepine receptors.

EP-A-0645378 also describes dihydro-1,3,4-benzotriazepines, which are said to inhibit squalene synthetase.

Dihydro-1,3,4-benzotriazepines in which the imine group within the 7-membered is reduced (tetrahydro-1,3,4-benzotriazepines) have been described in Morgenstern, et al., Pharmazie, (1996), 51(7), 458-467; Pflegel et al., Pharmazie, (1985), 40(10), 710-713; Pflegel et al., Pharmazie, (1982), 37(12), 829-832; and Pflegel et al., Pharmazie, (1980), 35(5/6), 332-333. However, these compounds do not fall within the scope of the present invention.

It is an object of the present invention to provide potent and selective gastrin and CCK receptor ligands.

According to the present invention, there are provided compounds of formula (I): 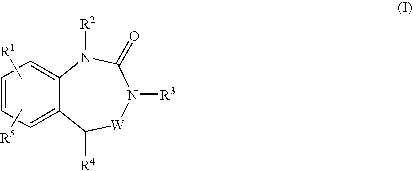
wherein:
W is NZ or NO;
R¹ and R⁵ are independently H, C₁ to C₆ alkyl, (C₁ to C₆ alkyl)oxy, thio, (C₁ to C₆ alkyl)oxycarbonyl, (C₁ to C₆ alkyl)carbonyloxy, nitro, trihalomethyl, hydroxy, hydroxy(C₁ to C₆ alkyl), amino, (C₁ to C₆ alkyl)amino, di(C₁ to C₆ alkyl)amino, aminocarbonyl, halo, halo(C₁ to C₆ alkyl), aminosulfonyl, (C₁ to C₆ alkyl)sulfonylamino, (C₁ to C₆ alkyl)aminocarbonyl, di(C₁ to C₆ alkyl)aminocarbonyl, [N-Z](C₁ to C₆ alkyl)carbonylamino, formyloxy, formamido, (C₁ to C₆ alkyl)aminosulfonyl, di(C₁ to C₆ alkyl)aminosulfonyl, [N-Z](C₁ to C₆ alkyl)sulfonylamino or cyano; or R¹ and R⁵ together form a methylenedioxy group;
R² is H or an optionally substituted C₁ to C₁₈ hydrocarbyl group wherein up to three C atoms may optionally be replaced by N, O and/or S atoms;
R³ is —(CR¹¹R¹²)ₘ—X—(CR¹³R¹⁴)ₚ—R⁹;
m is 0, 1, 2, 3 or 4;
p is 0, 1 or 2;
X is a bond, —CR¹⁵═CR¹⁶—, —C≡C—, C(O)NH, NHC(O), C(O)NMe, NMeC(O), C(O)O, NHC(O)NH, NHC(O)O, OC(O)NH, NH, O, CO, SO₂, SO₂NH, C(O)NHNH, 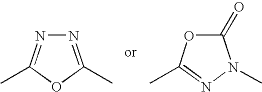
R⁹ is H; C₁ to C₆ alkyl; or phenyl, naphthyl, pyridyl, benzimidazolyl, indazolyl, quinolinyl, isoquinolinyl, tetrahydroisoquinolinyl, indolinyl, isoindolinyl, indolyl, isoindolyl or 2-pyridonyl, all optionally substituted with 1, 2 or 3 groups independently selected from -L-Q
   wherein:
L is a bond, or a group of the formula —(CR¹⁷R¹⁸)ᵥ—Y—(CR¹⁷R¹⁸)_{w}, wherein v and w are independently 0, 1, 2 or 3, and Y is a bond, —CR¹⁵═CR¹⁶—, phenyl, furanyl, thiophenyl, pyrrolyl, thiazolyl, imidazolyl, oxazolyl, isoxazolyl, pyrazolyl, isoxazolonyl, piperazinyl, piperidinyl, morpholinyl, pyrrolidinyl, isothiazolyl, triazolyl, oxadiazolyl, thiadiazolyl, pyridyl or pyridazyl; and
Q is H, (C₁ to C₆ alkyl)oxy, [N-Z](C₁ to C₆ alkyl)oxy(C₁ to C₆ alkyl)amino, thio, (C₁ to C₆ alkyl)thio, carboxy(C₁ to C₆ alkyl)thio, carboxy, carboxy(C₁ to C₆ alkyl), carboxy(C₁ to C₆ alkenyl), [N-Z]carboxy(C₁ to C₆ alkyl)amino, carboxy(C₁ to C₆ alkyl)oxy, formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, amino, [N-Z](C₁ to C₆ alkyl)amino, aminocarbonyl, (C₁ to C₆ alkyl)aminocarbonyl, di(C₁ to C₆ alkyl)aminocarbonyl, [N-Z](C₁ to C₆ alkyl)carbonylamino, C₅ to C₈ cycloalkyl, [N-Z](C₁ to C₆ alkyl)carbonyl(C₁ to C₆ alkyl)amino, halo, halo(C₁ to C₆ alkyl), sulfamoyl, [N-Z](C₁ to C₆ alkyl)sulfonylamino, (C₁ to C₆ alkyl)sulfonylaminocarbonyl, carboxy(C₁ to C₆ alkyl)sulfonyl, carboxy(C₁ to C₆ alkyl)sulfinyl, tetrazolyl, [N-Z]tetrazolylamino, cyano, amidino, amidinothio, SO₃H, formyloxy, formamido, C₃ to C₈ cycloalkyl, (C₁ to C₆ alkyl)sulphamoyl, di(C₁ to C₆ alkyl)sulphamoyl, (C₁ to C₆ alkyl)carbonylaminosulfonyl, 5-oxo-2,5-dihydro[1,2,4]oxadiazolyl, carboxy(C₁ to C₆ alkyl)carbonylamino, tetrazolyl(C₁ to C₆ alkyl)thio, [N-Z]tetrazolyl(C₁ to C₆ alkyl)amino, 5-oxo-2,5-dihydro[1,2,4]thiadiazolyl, 5-oxo-1,2-dihydro[1,2,4]triazolyl, [N-Z](C₁ to C₆ alkyl)amino(C₁ to C₆ alkyl)amino, or a group of the formula 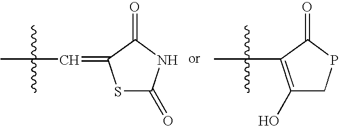
   wherein P is O, S or NR¹⁹;
Z is H, C₁ to C₆ alkyl, t-butoxycarbonyl, acetyl, benzoyl or benzyl;
R⁴ is an optionally substituted C₁ to C₁₈ hydrocarbyl group wherein up to three C atoms may optionally be replaced by N, O and/or S atoms;
R¹¹, R¹², R¹³, R¹⁴, R¹⁵, R¹⁷, R¹⁸ and R¹⁹ are independently H or C₁ to C₃ alkyl; and
R¹⁶ is H, C₁ to C₃ alkyl, or acetylamino;
or a pharmaceutically acceptable salt thereof
with the proviso that when R¹ is chloro, R² is H, R⁴ is phenyl and R⁵ is H; R³ may not be H, methyl or 2-hydroxyethyl.

Preferably, W is NH.

Preferably R¹ and R⁵ are both H. However, it will be appreciated the benzo-fused ring system may have one or two substituents on the benzene ring as indicated hereinabove. The substituents may have subtle steric and/or electronic effects which modify the activity of the compound at the gastrin receptor. However, the presence or otherwise of certain substituents on the benzene ring is not crucial to the overall pharmacological activity of the present compounds.

Preferably, R² is of formula:
—(CH₂)ₛ—C(R⁶R⁷)ₙ—(CH₂)ₜ—R⁸
wherein:
R⁶ and R⁷ are independently selected from H, C₁ to C₆ alkyl or OH; or R⁶ and R⁷ together represent an ═O group;
n is 0 or 1;
s is 0, 1, 2 or 3;
t is 0, 1, 2 or 3; and
R⁸ is selected from H, OH, C₁ to C₁₂ alkyl, (C₁ to C₁₂ alkyl)oxy, C₃ to C₁₂ cycloalkyl, phenyl, naphthyl, pyridyl, pyrrolyl, imidazolyl, pyrazolyl, pyridazinyl, pyrimidinyl, triazolyl, furanyl, thienyl, furazanyl, oxazolyl, isoxazolyl, thiazolyl, thiazinyl, indolyl, indolinyl, isoindolyl, isoindolinyl, isoquinolinyl, quinolinyl, benzofuranyl, benzothienyl, piperazinyl, piperidinyl, pyrrolidinyl, pyrrolinyl, dihydropyranyl, tetrahydropyranyl, pyranyl, tetrahydrofuranyl, morpholinyl, thiazolidinyl, thiomorpholinyl or thioxanyl (all optionally substituted with 1, 2 or 3 groups independently selected from C₁ to C₆ alkyl, (C₁ to C₆ alkyl)oxy, thio, (C₁ to C₆ alkyl)thio, carboxy, carboxy(C₁ to C₆ alkyl), formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, hydroxy(C₁ to C₆ alkyl), amino, (C₁ to C₆ alkyl)amino, di(C₁ to C₆ alkyl)amino, aminocarbonyl, halo, halo(C₁ to C₆ alkyl), aminosulfonyl, (C₁ to C₆ alkyl)sulfonylamino or cyano).

More preferably R² is of the formula:
—(CH₂)ₛ—C(O)—(CH₂)ₜ—R⁸
wherein:
s is 0, 1, 2 or 3;
t is 0, 1, 2 or 3;
R⁸ is selected from H, OH, C₁ to C₁₂ alkyl, (C₁ to C₁₂ alkyl)oxy, C₃ to C₁₂ cycloalkyl, phenyl, naphthyl, pyridyl, pyrrolyl, imidazolyl, pyrazolyl, pyridazinyl, pyrimidinyl, triazolyl, furanyl, thienyl, furazanyl, oxazolyl, isoxazolyl, thiazolyl, thiazinyl, indolyl, indolinyl, isoindolyl, isoindolinyl, isoquinolinyl, quinolinyl, benzofuranyl, benzothienyl, piperazinyl, piperidinyl, pyrrolidinyl, pyrrolinyl, dihydropyranyl, tetrahydropyranyl, pyranyl, tetrahydrofuranyl, morpholinyl, thiazolidinyl, thiomorpholinyl or thioxanyl (all optionally substituted with 1, 2 or 3 groups independently selected from C₁ to C₆ alkyl, (C₁ to C₆ alkyl)oxy, thio, (C₁ to C₆ alkyl)thio, carboxy, carboxy(C₁ to C₆ alkyl), formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, hydroxy(C₁ to C₆ alkyl), amino, (C₁ to C₆ alkyl)amino, di(C₁ to C₆ alkyl)amino, aminocarbonyl, halo, halo(C₁ to C₆ alkyl), aminosulfonyl, (C₁ to C₆ alkyl)sulfonylamino or cyano).

A further preferred group of compounds according to the present invention is where R² is of formula:
—(CH₂)C(O)R⁸
wherein:
R⁸ is a branched C₃ to C₁₂ alkyl group (such as tert-butyl, sec-butyl, isopropyl, isobutyl or isovaleryl); or R⁸ is a C₃ to C₁₂ cycloalkyl (such as cyclopentyl, cyclohexyl, cycloheptyl or adamantyl) phenyl, pyridyl, pyrrolidinyl or piperidinyl group (all optionally substituted with 1, 2 or 3 C₁₋₆ alkyl groups).

Preferably, R¹¹, R¹², R¹³, R¹⁴, R¹⁵, R¹⁶, R¹⁷, R¹⁸ and R¹⁹ are all H.

A preferred group of compounds according to the present invention is where R³ is of formula:
—(CH₂)—X—R⁹
wherein:
X is C(O)NH or NHC(O), more preferably X is C(O)NH.

Preferably, R⁹ is phenyl substituted with a carboxy, carboxy(C₁ to C₆ alkyl), tetrazolyl, tetrazolyl-N-(C₁ to C₆ alkyl)amino, carboxy(C₁ to C₆ alkyl)thio, carboxy(C₁ to C₆ alkyl)sulfonyl, (C₁ to C₆ alkyl)amino, or 5-oxo-2,5-dihydro[1,2,4]oxadiazolyl group; or R⁹ is a N-[carboxy(C₁ to C₆ alkyl)]indolinyl or N-[carboxy(C₁ to C₆ alkyl)]indolyl group.

When R⁹ is a substituted phenyl group, the substituent is preferably at the 3-position of the phenyl group.

Preferably, in compounds according to the present invention R⁴ is of formula:
—(CH₂)_{q}-T-R¹⁰
wherein:
q is 0, 1, 2 or 3;
T is a bond, O, S, NH or N(C₁ to C₆ alkyl); and
R¹⁰ is C₁ to C₁₂ alkyl, C₃ to C₁₂ cycloalkyl, phenyl, naphthyl, pyridyl, pyrrolyl, imidazolyl, pyrazolyl, pyridazinyl, pyrimidinyl, triazolyl, furanyl, thienyl, furazanyl, oxazolyl, isoxazolyl, thiazolyl, thiazinyl, indolyl, indolinyl, isoindolyl, isoindolinyl, isoquinolinyl, quinolinyl, benzofuranyl, benzothienyl, piperazinyl, piperidinyl, pyrrolidinyl, pyrrolinyl, dihydropyranyl, tetrahydropyranyl, pyranyl, tetrahydrofuranyl, morpholinyl, thiazolidinyl, thiomorpholinyl or thioxanyl (all optionally substituted with 1, 2 or 3 groups independently selected from C₁ to C₆ alkyl, (C₁ to C₆ alkyl)oxy, C₃ to C₈ cycloalkyl, (C₃ to C₈ cycloalkyl)oxy, thio, (C₁ to C₆ alkyl)thio, carboxy, carboxy(C₁ to C₆ alkyl), formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, hydroxy(C₁ to C₆ alkyl), amino, (C₁ to C₆ alkyl)amino, di(C₁ to C₆ alkyl)amino, aminocarbonyl, halo, halo(C₁ to C₆ alkyl), aminosulfonyl, (C₁ to C₆ alkyl)sulfonylamino or cyano).

More preferably, in compounds according to the present invention, R⁴ is selected from C₁₋₁₂ alkyl (such as tert-butyl, sec-butyl, isopropyl, isobutyl or isovaleryl), C₃₋₁₂ cycloalkyl (such as cyclopentyl, cyclohexyl, cycloheptyl or adamantyl), pyridyl or phenyl (all of which may be optionally substituted with 1, 2 or 3 groups selected from OMe, NMe₂, CF₃, Me, F, Cl, Br or I).

In all compounds of the present invention, preferably q is 0 and T is a bond. More preferably R⁴ is C₃-C₁₂ cycloalkyl, and more preferably, R⁴ is cyclohexyl.

Certain compounds of the invention exist in various regioisomeric, enantiomeric, tautomeric and diastereomeric forms. It will be understood that the invention comprehends the different regioisomers, enantiomers, tautomers and diastereomers in isolation from each other as well as mixtures.

### General Synthesis of tetrahydro-1,3,4-benzotriazepines

Compounds of the present invention wherein W is NH may be prepared by the representative procedure shown in Reaction Scheme 1.

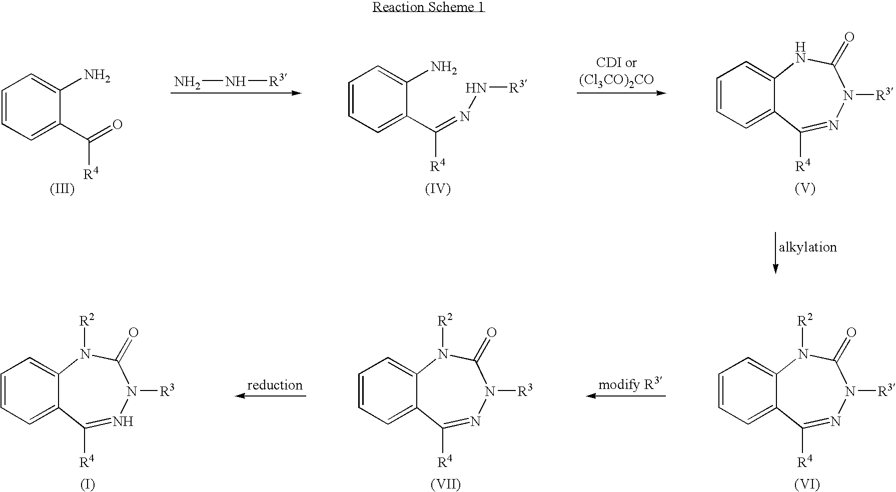

Ketone (III) is reacted with NH₂NHR^{3′} (wherein R^{3′} represents either R³ or a suitable precursor thereof) to form hydrazone (IV). The hydrazone (IV) is then cyclised using a bifunctional carbonyl reagent to form benzotriazepinone (V). Bifunctional carbonyl reagents are well known to the person skilled in the art and include, for example, carbonyldiimidazole (CDI), triphosgene, phosgene or cyanogen bromide. Alkylation under standard conditions followed by modification of R^{3′} affords the benzotriazepinone (VII). This is followed by a final reduction using 10% Pd on C and hydrogen to yield the desired tetrahydro-1,3,4-benzotriazepinone.

Compounds wherein W is NO may be prepared, for example, by treating compound I with an oxidising agent such as MCPBA.

Compounds of the invention are chiral by virtue of the carbon atom at position 5 of the ring. Separate enantiomers, such as those exemplified by examples 11, 12, 13 and 14 (see below), may be synthesised according to scheme 2. This is generalised for R³=(CR¹¹R¹²)m-CONR¹⁵—(CR¹³R¹⁴)p-R⁹. R*OH is a chiral alcohol such as menthol.

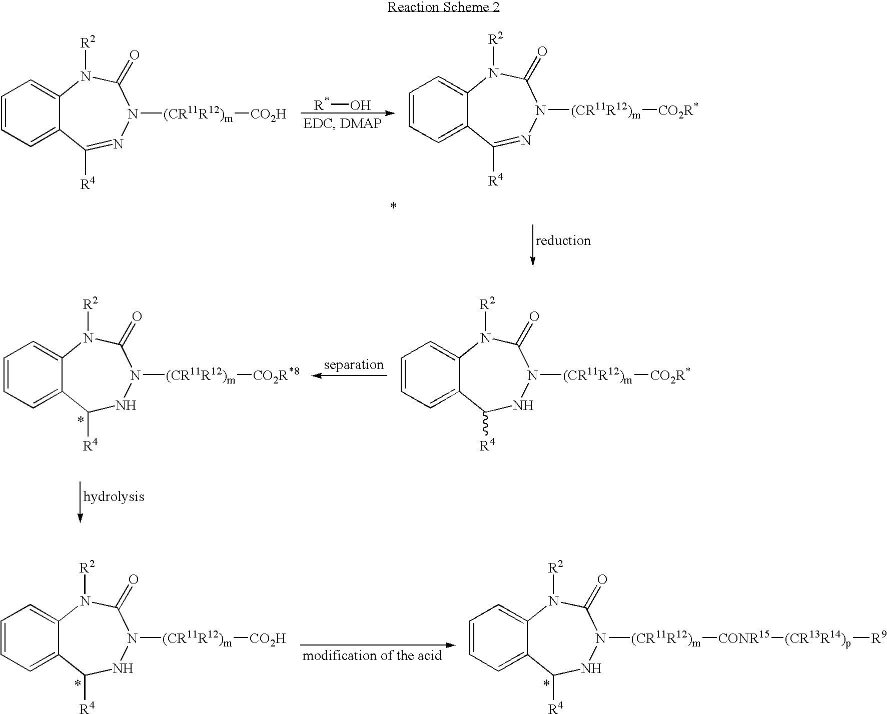

R^{3′} groups which are suitable precursors of R³ will depend on the particular nature of R³. For example, when R³ is —(CH₂)ₘC(O)NH—(CH₂)ₚ—R⁹, a suitable R^{3′} group would be —(CH₂)ₘCO₂(C₁₋₆ alkyl). In this case, the requisite R^{3′} groups may be readily accessed via an ester hydrolysis followed by a simple amide coupling reaction. Further deprotection, if appropriate, may be necessary to reveal the final R³ group. The skilled person will be aware of many other suitable R^{3′} groups, depending on the nature of R³.

Alkylation of compound V may be performed by, for example, displacement of an alkyl halide in the presence of a base. Methods of alkylation will be readily apparent to the person skilled in the art.

Hence, the present invention also provides a method of making compounds according to formula (I).

The invention also comprehends derivative compounds (“pro-drugs”) which are degraded in vivo to yield the species of formula (1) Pro-drugs are usually (but not always) of lower potency at the target receptor than the species to which they are degraded. Pro-drugs are particularly useful when the desired species has chemical or physical properties which make its administration difficult or inefficient. For example, the desired species may be only poorly soluble, it may be poorly transported across the mucosal epithelium, or it may have an undesirably short plasma half-life. Further discussion of pro-drugs may be found in Stella, V. J. et al., “Prodrugs”, Drug Delivery Systems, 1985, pp. 112-176, and Drugs, 1985, 29, pp. 455-473.

Pro-drug forms of the pharmacologically-active compounds of the invention will generally be compounds according to formula (I) having an acid group which is esterified or amidated. Included in such esterified acid groups are groups of the form —COOR^{a}, wherein R^{a} is C₁ to C₅ alky, phenyl, substituted phenyl, benzyl, substituted benzyl, or one of the following:

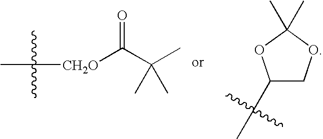

Amidated acid groups include groups of the formula —CONR^{b}R^{c}, wherein R^{b} is H, C₁ to C₅ alkyl, phenyl, substituted phenyl, benzyl, or substituted benzyl, and R^{c} is —OH or one of the groups just recited for R^{b}.

Compounds of formula (I) having an amino group may be derivatised with a ketone or an aldehyde such as formaldehyde to form a Mannich base. This will hydrolyse with first order kinetics in aqueous solution.

Another aspect of the present invention is a pharmaceutical composition comprising a compound of formula (I) substantially as described herein before with a pharmaceutically acceptable diluent or carrier.

Another aspect of the present invention is a compound of formula (I) or a pharmaceutical composition comprising a compound of formula (I) for use in medicine.

Another aspect of the present invention is a compound of formula (I) or a pharmaceutical composition comprising a compound of formula (I) for use in the preparation of a medicament for the treatment of gastrin related disorders.

Typical gastrin related disorders are gastrointestinal ulcers, dyspepsia, reflux oesophagitis (gastroesophageal reflux disease (GERD), both erosive and non-erosive), Zollinger-Ellison syndrome, Barrett's oesophagus (specialized intestinal metaplasia of distal oesophagus), ECL cell hyperplasia, rebound hypersecretion (following cessation of anti-secretory therapy), ECL-derived gastric polyps, cancers of the GI tract, more particularly in the stomach, oesophagus and colo-rectal areas, as well as tumours found in other organs such as the pancreas, lung (small cell lung carcinomas) and thyroid (thyroid medullary tumours) and anxiety. The potentiation of opiate induced analgesia may also provide a role for the gastrin ligands of the present invention.

Yet another aspect of the present invention is a method of making a pharmaceutical composition comprising a compound of formula (I) substantially as described herein before, comprising mixing said compound with a pharmaceutically acceptable diluent or carrier.

Pharmaceutically acceptable salts of the acidic or basic compounds of the invention can of course be made by conventional procedures, such as by reacting the free base or acid with at least a stoichiometric amount of the desired salt-forming acid or base.

Pharmaceutically acceptable salts of the acidic compounds of the invention include salts with inorganic cations such as sodium, potassium, calcium, magnesium, zinc, and ammonium, and salts with organic bases. Suitable organic bases include N-methyl-D-glucamine, arginine, benzathine, diolamine, olamine, procaine, chlorine and tromethamine.

Pharmaceutically acceptable salts of the basic compounds of the invention include salts derived from organic or inorganic acids. Suitable anions include acetate, adipate, besylate, bromide, camsylate, chloride, citrate, edisylate, estolate, fumarate, gluceptate, gluconate, glucuronate, hippurate, hyclate, hydrobromide, hydrochloride, iodide, isethionate, lactate, lactobionate, maleate, mesylate, methylbromide, methylsulfate, napsylate, nitrate, oleate, pamoate, phosphate, polygalacturonate, stearate, succinate, sulfate, sulfosalicylate, tannate, tartrate, terephthalate, tosylate and triethiodide.

It is anticipated that the compounds of the invention can be administered by oral or parenteral routes, including intravenous, intramuscular, intraperitoneal, subcutaneous, rectal and topical administration, and inhalation.

For oral administration, the compounds of the invention will generally be provided in the form of tablets or capsules or as an aqueous solution or suspension.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate and lactose. Corn starch and alginic acid are suitable disintegrating agents. Binding agents may include starch and gelatine. The lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatine capsules in which the active ingredient is mixed with a solid diluent and soft gelatine capsules wherein the active ingredient is mixed with water or an oil such as peanut oil, liquid paraffin or olive oil.

For intramuscular, intraperitoneal, subcutaneous and intravenous use, the compounds of the invention will generally be provided in sterile aqueous solutions or suspensions, buffered to an appropriate pH and isotonicity. Suitable aqueous vehicles include Ringer's solution and isotonic sodium chloride. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate.

Effective doses of the compounds of the present invention may be ascertained be conventional methods. The specific dosage level required for any particular patient will depend on a number of factors, including severity of the condition being treated, the route of administration and the weight of the patient. In general, however, it is anticipated that the daily dose (whether administered as a single dose or as divided doses) will be in the range 0.001 to 5000 mg per day, more usually from 1 to 1000 mg per day, and most usually from 10 to 200 mg per day. Expressed as dosage per unit body weight, a typical dose will be expected to be between 0.01 μg/kg and 50 mg/kg, especially between 10 μg/kg and 10 mg/kg, eg. between 100 μg/kg and 2 mg/kg.

In a further aspect of the present invention there are provided pharmaceutical compositions comprising a compound according to formula (I) and a proton pump inhibitor. Compositions comprising a CCK₂/gastrin antagonist and a proton pump inhibitor are described in International patent application WO93/12817, incorporated herein by reference.

In one aspect of the present invention the proton pump inhibitor is
omeprazole which is 5-methoxy-2-[[(4-methoxy-3,5-dimethyl-2-pyridinyl)-methyl]sulfinyl]-1H-benzimidazole;
BY308;
SK&F 95601 which is 2-[[(3-chloro-4-morpholino-2-pyridyl)methyl]sulfinyl]-5-methoxy-(1H)-benzimidazole;
SK & 96067 which is 3-butyryl-4-(2-methylphenylamino)-8-methoxyquinoline;
5-trifluoromethyl-2-[4-methoxy-3-methyl-2-pyridyl-methyl]-thio-[1H]-benzimidazole;
   or pharmaceutically acceptable salts thereof.

These proton pump inhibitors are described and claimed in U.S. Pat. Nos. 4,472,409 and 4,255,431. These patents are incorporated herein by reference.

In a further aspect of the present invention, the proton pump inhibitor is
lansoprazole which is 2-[[[3-methyl-4-(2,2,2-trifluoroethoxy)-2-pyridinyl]methyl]sulfinyl]-1H-benzimidazole;
pantoprazole which is 5-(difluoromethoxy)-2-[[(3,4-dimethoxy-2-pyridinyl)methyl]sulfinyl]-1H-benzimidazole;
perprazole;
rabeprazole which is 2-[[4-(3-methoxypropoxy)-3-methylpyridin-2-yl]methylsulfinyl]-1H-benzimidazole;
[[4-(2,2,2-trifluoroethoxy)-3-methyl-2-pyridyl]-methyl]sulfenamide;
(Z)-5-methyl-2-[2-(1-naphthyl)ethenyl]4-piperidinopyridine HCl;
2-(4-cyclohexyloxy-5-methylpyridin-2-yl)-3-(1-naphthyl)-1-propanol;
methyl 2-cyano-3-(ethylthio)-3-(methylthio)-2propenoate;
2-((4-methoxy-2-pyridyl)methylsulphinyl)-5-(1,1,2,2-tetrafluoroethoxy)-1H-benzimidazole sodium;
2-[[[4-(2,2,3,3,4,4,4-heptafluorobutoxy)-2-pyridyl]methyl)sulfinyl]-1H-thieno[3,4-d]imidazole;
2-[[[4-(2,2,2-trifluoroethoxy)-3-methyl-2-pyridyl]methyl]sulfinyl]-1H-benzimidazole;
2-[[[4-(2,2,2-trifluoroethoxy)-3-methyl-2-pyridyl]methyl]sulfinyl]-1H-benzimidazole;
2-methyl-8-(phenylmethoxy)-imidazo(1,2-A)-pyridine-3-acetonitrile;
(2-((2-dimethylaminobenzyl)sulfinyl)-benzimidazole);
4-(N-allyl-N-methylamino)-1-ethyl-8-((5-fluoro-6-methoxy-2-benzimidazolyl)sulfinylmethyl)-1-ethyl 1,2,3,4-tetrahydroquinolone;
2-[[(2-dimethylaminophenyl)methyl]sulfinyl]-4,7-dimethoxy-1H-benzimidazole;
2-[(2-(2-pyridyl)phenyl)sulfinyl)-1H-benzimidazole;
(2-[(2-amino-4-methylbenzyl)sulfinyl]-5-methoxybenzo[d]imidazole;
(4(2-methylpyrrol-3-yl)-2-guanidisothiazole);
4-(4-(3-(imidazole)propoxy)phenyl)-2phenylthiazole;
(E)-2-(2-(4-(3-(dipropylamino)butoxy)phenyl)-ethenyl)benzoxazole;
(E)-2-(2-(4-(3-(dipropylamino)propoxy)phenyl)ethenyl)-benzothiazole;
Benzeneamine, 2-[[(5-methoxy-1H-benzimidazol-2-yl)sulfinyl]methyl)-4-methyl-;
Pumilacidin A;
2,3-dihydro-2-methoxycarbonylamino-1,2-benzisothiazol-3-one;
2-(2-ethylaminophenylmethylsulfinyl)-5,6-dimethoxybenzimidazole;
2-methyl-8-(phenylmethoxy)imidazo[1,2-a)pyridine-3-acetonitrile;
3-amino-2-methyl-8-phenylmethoxyimidazo[1,2-a)-pyrazine HCl;
2-[[(3-chloro-4-morpholino-2-pyridyl)methyl]-sulfinyl)-5-methoxy-(1H)-benzinidazole;
[3-butyryl-4-(2-methylphenylamino)-8-methoxy-quinoline);
2-indanyl 2-(2-pyridyl)-2-thiocarbamoylacetate HCl;
2,3-dihydro-2-(2-pyridinyl)-thiazolo (3,2-a)-benzimidazole;
3-cyanomethyl-2-methyl-8-(3-methyl-2-butenyloxy)-(1,2-a)imidazopyridine;
zinc L-carnosine;
   or pharmaceutically acceptable salts thereof.

Rabeprazole is described in U.S. Pat. No. 5,045,552. Lansoprazole is described in U.S. Pat. No. 4,628,098. Pantoprazole is described in U.S. Pat. No. 4,758,579. These patents are incorporated herein by reference.

Preferably, the proton pump inhibitor is selected from (RS)-rabeprazole, (RS)-omeprazole, lansoprazole, pantoprazole, (R)-omeprazole, (S)-omeprazole, perprazole, (R)-rabeprazole, (S)-rabeprazole, or the alkaline salts thereof. The alkaline salts may be, for example, the lithium, sodium, potassium, calcium or magnesium salts.

Compositions of this invention comprising a compound of formula (I) and a proton pump inhibitor may be administered as described above. Preferably the dose of each of the active ingredients in these compositions will be equal to or less than that which is approved or indicated in monotherapy with said active ingredient.

In another aspect of this invention, there is provided a kit comprising a compound of formula (I) and a proton pump inhibitor. The kit is useful as a combined preparation for simultaneous, separate or sequential use in the treatment of patients suffering from gastrointestinal disorders.

In yet a further aspect of the present invention there is provided a method of making a pharmaceutical composition comprising a compound of formula (I) substantially as described herein before and a proton pump inhibitor, comprising mixing said compound and said proton pump inhibitor with a pharmaceutically acceptable carrier or diluent.

The term “hydrocarbyl” is used herein to refer to monovalent groups consisting of carbon and hydrogen. Hydrocarbyl groups thus include alkyl, alkenyl and alkynyl groups (in both straight and branched chain forms), cycloalkyl (including polycycloalkyl groups such as bicyclooctyl and adamantyl), cycloalkenyl and aryl groups, and combinations of the foregoing, such as alkylcycloalkyl, alkylpolycycloalkyl, alkylaryl, alkenylaryl, alkynylaryl, cycloalkylaryl and cycloalkenylaryl groups.

Where reference is made to a carbon atom of a hydrocarbyl group being replaced by a N, O or S atom, what is intended is that 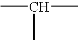
is replaced by 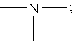
or that —CH₂— is replaced by —O— or —S—.

Where reference is made to an optionally substituted hydrocarbyl group, the hydrocarbyl group is substituted with 1, 2 or 3 groups independently selected from -L-Q

wherein:

L is a bond, or a group of the formula —(CR¹⁷R¹⁸)ᵥ—Y—(CR¹⁷R¹⁸)_{w}, wherein v and w are independently 0, 1, 2 or 3, and Y is a bond, —CR¹⁵═CR¹⁶—, phenyl, furanyl, thiophenyl, pyrrolyl, thiazolyl, imidazolyl, oxazolyl, isoxazolyl, pyrazolyl, isoxazolonyl, piperazinyl, piperidinyl, morpholinyl, pyrrolidinyl, isothiazolyl, triazolyl, oxadiazolyl, thiadiazolyl, pyridyl or pyridazyl;
Q is H, (C₁ to C₆ alkyl)oxy, [N-Z](C₁ to C₆ alkyl)oxy(C₁ to C₆ alkyl)amino, thio, (C₁ to C₆ alkyl)thio, carboxy(C₁ to C₆ alkyl)thio, carboxy, carboxy(C₁ to C₆ alkyl), carboxy(C₁ to C₆ alkenyl), [N-Z]carboxy(C₁ to C₆ alkyl)amino, carboxy(C₁ to C₆ alkyl)oxy, formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, amino, [N-Z](C₁ to C₆ alkyl)amino, aminocarbonyl, (C₁ to C₆ alkyl)aminocarbonyl, di(C₁ to C₆ alkyl)aminocarbonyl, [N-Z](C₁ to C₆ alkyl)carbonylamino, C₅ to C₈ cycloalkyl, [N-Z](C₁ to C₆ alkyl)carbonyl(C₁ to C₆ alkyl)amino, halo, halo(C₁ to C₆ alkyl), sulfamoyl, [N-Z](C₁ to C₆ alkyl)sulfonylamino, (C₁ to C₆ alkyl)sulfonylaminocarbonyl, carboxy(C₁ to C₆ alkyl)sulfonyl, carboxy(C₁ to C₆ alkyl)sulfinyl, tetrazolyl, [N-Z]tetrazolylamino, cyano, amidino, amidinothio, SO₃H, formyloxy, formamido, C₃ to C₈ cycloalkyl, (C₁ to C₆ alkyl)sulphamoyl, di(C₁ to C₆ alkyl)sulphamoyl, (C₁ to C₆ alkyl)carbonylaminosulfonyl, 5-oxo-2,5-dihydro[1,2,4]oxadiazolyl, carboxy(C₁ to C₆ alkyl)carbonylamino, tetrazolyl(C₁ to C₆ alkyl)thio, [N-Z]tetrazolyl(C₁ to C₆ alkyl)amino, 5-oxo-2,5-dihydro[1,2,4]thiadiazolyl, 5-oxo-1,2-dihydro[1,2,4]triazolyl, [N-Z](C₁ to C₆ alkyl)amino(C₁ to C₆ alkyl)amino, or a group of the formula 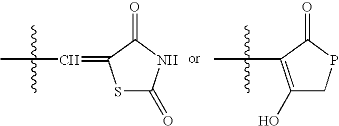
   wherein P is O, S or NR¹⁹;

and
Z is H, C, to C₆ alkyl, t-butoxycarbonyl, acetyl, benzoyl or benzyl.

The term “alkyl” is used herein to refer to both straight and branched chain forms. Further, the alkyl chain may include multiple bonds. Hence, the term “alkyl” also encompasses alkenyl and alkynyl groups. Likewise, the term “cycloalkyl” also encompasses cycloalkenyl groups. Preferably, alkyl and cycloalkyl groups as used in the present invention do not contain multiple bonds. Where there are preferred alkenyl groups, these are specified as alkenyl groups. However, specific reference to alkenyl groups is not to be construed as any limitation on the definition of alkyl groups as described above.

Where reference is made to dialkyl groups [e.g. di(C₁ to C₆ alkyl)amino groups], it is understood that the two alkyl groups may be the same or different.

In the interests of simplicity, terms which are normally used to refer to monovalent groups (such as “alkyl” or “phenyl”) are also used herein to refer to divalent bridging groups which are formed from the corresponding monovalent group by the loss of one hydrogen atom. Whether such a term refers to a monovalent group or to a divalent group will be clear from the context. For example, when L is —(CR¹⁷R¹⁸)ᵥ—Y—(CR¹⁷R¹⁸)_{w}—, it is clear that Y must be a divalent group. Thus, when Y is defined as thiazolyl, for example, this refers to a divalent group having the structure

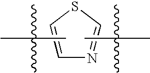

Where, as in this example, a divalent bridging group is formed from a cyclic moiety, the linking bonds may be on any suitable ring atom, subject to the normal rules of valency. Accordingly, by way of further example, the term pyrrolyl in the definition of Y includes all of the following groups:

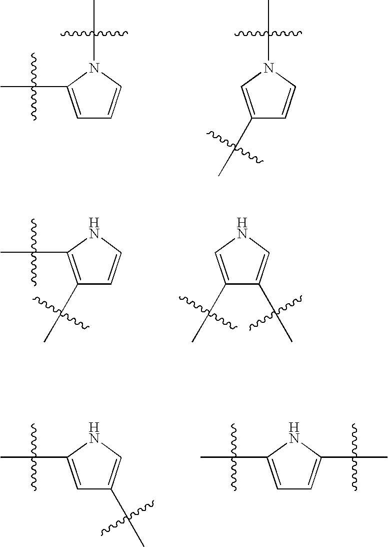

The term “halogen” or “halo” is used herein to refer to any of fluorine, chlorine, bromine and iodine. Most usually, however, halogen substituents in the compounds of the invention are chlorine and fluorine substituents. Groups such as halo(C₁ to C₆ alkyl) includes mono-, di- or tri-halo substituted C₁ to C₆ alkyl groups. Moreover, the halo substitution may be at any position in the alkyl chain.

The prefix [N-Z] refers to possible substitution of an amino group in the following compound or substituent name. For example, [N-Z]alkylamino refers to groups of the form

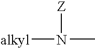

Similarly, [N-Z]tetrazolylamino, wherein Z is C₁ to C₆ alkyl, includes groups such as tetrazolyl[N-methyl]amino and tetrazolyl[N-ethyl]amino. Of course, when Z is H, no substitution is present.

In case there is any doubt, the group named as 5-oxo-2,5-dihydro[1,2,4]oxadiazolyl has the following formula 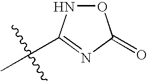
and comprehends tautomeric forms.

The invention is now further illustrated by means of the following Examples.

### EXPERIMENTAL

Commercially available dichloromethane (DCM), tetrahydrofuran (THF) and N,N-dimethylformamide (DMF) were used. Flash column chromatography was performed on Merck silica gel 60 (40-63 μm) using the reported solvent systems. ¹H NMR spectra were recorded on a Bruker DRX-300 instrument at 300 MHz and the chemical shifts (δ_{H}) were recorded relative to an internal standard. (2-Amino-phenyl)-cyclohexyl-methanone was prepared by a published method (M. S. Chambers, et. al., Bioorg. Med. Chem. Lett. (1993), 3, 1919). (2-Amino-phenyl)-cycloheptyl-methanone was prepared according to the method of A. Cappelli, et. al., J. Med. Chem., (1999), 42, 1556). 2-Bromo-1-cyclopentyl-ethanone and 2-bromo-1-cyclohexyl-ethanone were prepared by a published method (M. Gaudry, A. Marquet, Org. Synth., (1976), 55, 24). Substituted anilines, which are not available commercially and whose preparation is not described in detail, were prepared from commercially available starting materials, using standard chemical transformations.

### Example 1

### (+/−)-3-{2-[5-Cyclohexyl-1-(3,3-dimethyl-2-oxo-butyl)-2-oxo-1,2,4,5-tetrahydro-3H-1,3,4-benzotriazepin-3-yl]-acetylamino)-benzoic acid

Step a. {N′-[(2-Amino-phenyl)-cyclohexyl-methylene]-hydrazino)-acetic acid ethyl ester. A mixture of (2-amino-phenyl)-cyclohexyl-methanone (20.3 g, 0.1 mol) and ethyl hydrazinoacetate hydrochloride (23.25 g, 0.15 mol) and pyridine (12.1 ml, 0.15 mol) was heated at reflux under argon in EtOH (400 ml) for 72 h. On cooling, un-reacted ethyl hydrazinoacetate hydrochloride crystallised from the solution and was removed by filtration. The filtrate was evaporated and the residue was partitioned between saturated NaHCO₃ (250 ml) and EtOAc (250 ml). The organic phase was washed with brine (250 ml), dried over MgSO₄ then the solvent was evaporated under reduced pressure. The residue was purified by flash column chromatography (EtOAc-hexane (1:4)) to afford {N′-[(2-amino-phenyl)-cyclohexyl-methylene]-hydrazino}-acetic acid ethyl ester as a pale yellow foam (21.2 g, 71%) and un-reacted (2-amino-phenyl)-cyclohexyl-methanone (2.40 g). ¹H NMR (CDCl₃) 7.17 (1H, dt), 6.98 (1H, dd), 6.80 (1H, dt), 6.73 (1H, dd), 5.32 (1H, t), 4.16 (2H, m), 3.95 (2H, br s), 3.89 (2H, m), 2.37 (1H, m), 1.80 (1H, m), 1.75-1.61 (4H, m), 1.33-1.19 (8H, m).

Step b. (S-Cyclohexyl-2-oxo-1,2-dihydro-3H-1,3,4-benzotriazepin-3-yl)-acetic acid ethyl ester. A solution of the product of step a (23.39 g, 77.0 mmol) and triethylamine (26.8 ml, 0.19 mol) in DCM (300 ml) was stirred at 0° C. under argon and a solution of triphosgene (11.4 g, 39 mmol) in DCM (100 ml) was added drop-wise over 1 h. The reaction mixture was stirred at this temperature for 1 h, washed with H₂O (300 ml), saturated NaHCO₃ (300 ml) and brine (300 ml). The organic phase was dried over MgSO₄, filtered and the solvent was evaporated under reduced pressure. The crude product was re-crystallised from Et₂O-hexane (1:3) to afford (5-cyclohexyl-2-oxo-1,2-dihydro-3H-1,3,4-benzotriazepin-3-yl)-acetic acid ethyl ester (15.8 g, 62%), as a yellow solid. ¹H NMR (CDCl₃) 7.35 (2H, m), 7.12 (1H, t), 6.85 (2H, m), 4.32 (2H, s), 4.18 (2H, m), 2.68 (1H, m), 1.81-1.68 (5H, m), 1.49-1.22 (8H, m).

Step c. [5-Cyclohexyl-1-(3,3-dimethyl-2-oxo-butyl)-2-oxo-1,2-dihydro-3H-1,3,4-benzotriazepin-3-yl]-acetic acid ethyl ester. To an ice-cooled solution of the product of step b (3.29 g, 10.0 mmol) in DMF (30 ml) was added sodium hydride (60% dispersion in mineral oil, 480 mg, 12.0 mmol) in small portions. The mixture was stirred at room temperature for 30 min then 1-bromo-3,3-dimethyl-butan-2-one (1.60 ml, 12.0 mmol) was added. The reaction mixture was stirred at room temperature for 2 h, diluted with H₂O (200 ml) and extracted with EtOAc (30 ml×3). The combined organic extracts were washed with brine (50 ml), dried over MgSO₄, filtered and the solvent was evaporated under reduced pressure. The residue was purified by flash column chromatography (EtOAc-DCM (1:9)) to afford the product as a yellow foam (3.59 g, 84%). ¹H NMR (CDCl₃) 7.37 (2H, m), 7.17 (1H, dt), 6.93 (1H, d), 4.66 (2H, s), 4.35 (1H, m), 2.74 (1H, m), 1.90-1.70 (6H, m), 1.31-1.16 (16H, m).

Step d. [5-Cyclohexyl-1-(3,3-dimethyl-2-oxo-butyl)-2-oxo-1,2-dihydro-3H-1,3,4-benzotriazepin-3-yl]-acetic acid. A solution of the product of step c (3.57 g, 8.20 mmol), and 1.0M NaOH (8.70 ml, 8.70 mmol) in EtOH (30 ml) was stirred at room temperature for 16 h. The EtOH was evaporated under reduced pressure, the residue was diluted with H₂O (30 ml) and acidified to pH 3 with 1N HCl. The mixture was extracted with DCM (30 ml×2), and the combined extracts were dried over MgSO₄. Filtration and evaporation of the solvent under reduced pressure afforded the product as a pale yellow foam (3.10 g, 95%). ¹H NMR (CDCl₃) 11.00 (1H, br s), 7.45 (2H, m), 7.25 (1H, m), 6.97 (1H, dd), 4.68 (2H, m), 4.25 (1H, d), 3.90 (1H, d), 2.80 (1H, m), 2.08-1.61 (6H, m), 1.44-1.18 (13H, m).

Step e. 3-{2-[5-Cyclohexyl-1-(3,3-dimethyl-2-oxo-butyl)-2-oxo-1,2-dihydro-3H-1,3,4-benzotriazepin-3-yl]-acetylamino}-benzoic acid benzyl ester. To a solution of the product of step d (410 mg, 1.00 mmol), and 3-amino-benzoic acid benzyl ester (230 mg, 1.00 mmol) in DMF (10 ml) was added 1-hydroxybenzotriazole (HOBt) (200 mg, 1.50 mmol), 4-dimethylaminopyridine (DMAP) (12 mg, 0.10 mmol) and 1-(3-dimethylaminopropyl)-3-ethylcarbodiimide hydrochloride (EDC) (290 mg, 1.50 mmol). The solution was maintained at room temperature under argon for 16 h, diluted with H₂O (25 ml) and the reaction mixture was extracted with EtOAc (20 ml×2). The combined extracts were washed with 5% KHSO₄ (20 ml), saturated NaHCO₃ (20 ml) and brine (20 ml×3). The organic phase was dried over MgSO₄. Filtration and evaporation of the solvent gave the crude product, which was purified by flash column chromatography (Hexane-EtOAc (7:3)) to afford the title compound, as a colourless foam (358 mg, 59%). ¹H NMR (CDCl₃) 8.41 (1H, s), 7.98 (1H, m), 7.77 (2H, m), 7.46-7.34 (8H, m), 7.20 (1H, t), 7.00 (1H, d), 5.36 (2H, s), 4.74 (1H, d), 4.61 (1H, d), 4.30 (1H, d), 4.20 (1H, d), 2.77 (1H, m), 2.02-1.62 (6H, m), 1.35-1.22 (13H, m).

Step f. A round bottom flask containing the product of step e (358 mg, 0.60 mmol), 10% palladium on charcoal (40 mg) and THF-MeOH (1:1/20 ml) was evacuated and flushed with hydrogen three times. The mixture was stirred vigorously overnight under an atmosphere of hydrogen. The catalyst was removed by filtration through a pad of celite and the filtrate evaporated to afford the product as a white solid (280 mg, 91%). ¹H NMR (CDCl₃) 8.66 (1H, s), 8.28 (1H, d), 8.14 (1H, s), 7.85 (1H, d), 7.46 (1H, t), 7.27-7.20 (3H, m), 6.98 (1H, d), 5.30 (1H, br s), 5.18 (1H, d), 4.40-4.27 (3H, m), 3.97 (1H, d), 1.73-1.59 (5H, m), 1.33-1.06 (15H, m). The compound was further characterised as the N-methyl-D-glucamine salt. Found: C, 57.70, H, 7.18, N, 9.11%; C₂₉H₃₆N₄O₅.C₇H₁₇NO₅.0.5CH₂Cl₂ requires: C, 57.81, H, 7.18, N, 9.24%.

### Example 2

### (+/−)-2-[5-Cyclohexyl-1-(3,3-dimethyl-2-oxo-butyl)-2-oxo-1,2,4,5-tetrahydro-3H-1,3,4-benzotriazepin-3-yl]-N-(3-methylamino-phenyl)-acetamide

Step a. (3-Nitro-phenyl)-carbamic acid tert-butyl ester. A solution of 3-nitrophenyl isocyanate (14.44 g, 88.0 mmol) in tert-butanol (80 ml) was heated at reflux under argon for 2 h. After cooling the solvent was evaporated, the residue was dried under high vacuum, washed thoroughly with Et₂O to afford the product as a yellow solid (19.96 g, 95%). ¹H NMR (CDCl₃) 8.30 (1H, s), 7.88 (1H, d), 7.71 (1H, d), 7.45 (1H, t), 6.68 (1H, br s), 1.55 (9H, s).

Step b. Methyl-(3-nitro-phenyl)-carbamic acid tert-butyl ester. An ice-cooled solution of the product of step a (3.57 g, 15.0 mmol) in DMF (30 ml) was stirred under argon and sodium hydride (60% dispersion in mineral oil, 720 mg, 18.0 mmol) was added in small portions. After stirring at room temperature for 1 h, the reaction mixture was cooled externally with ice and iodomethane (1.4 ml, 22.5 mmol) was added. The reaction mixture was stirred at room temperature for 2 h, H₂O (150 ml) was added and extracted with EtOAc (50 ml×2). The combined extracts were washed with brine, dried (MgSO₄), filtered and the solvent was evaporated. The residue was purified by flash column chromatography (EtOAc-DCM (1:9)) to afford the product as a yellow foam (3.34 g, 88%). ¹H NMR (CDCl₃) 8.16 (1H, t), 8.00 (1H, m), 7.63 (1H, m), 7.48 (1H, t), 3.34 (3H, s), 1.49 (9H, s).

Step c. (3-Amino-phenyl)-methyl-carbamic acid tert-butyl ester. A round bottom flask containing the product of step b (3.30 g, 13.1 mmol), 10% palladium on charcoal (300 mg) and THF-MeOH (1:1/50 ml) was evacuated and flushed with hydrogen three times. The mixture was stirred vigorously overnight under an atmosphere of hydrogen. The catalyst was removed by filtration through a pad of celite and the filtrate evaporated to afford the product as a white solid (2.90 g, 99%). ¹H NMR (CDCl₃) 7.10 (1H, t), 6.62 (2H, m), 6.50 (1H, m), 3.66 (2H, br s), 3.22 (3H, s), 1.46 (9H, s).

Step d. (3-{2-[5-Cyclohexyl-1-(3,3-dimethyl-2-oxo-butyl)-2-oxo-1,2-dihydro-3H-1,3,4-benzotriazepin-3-yl]-acetylamino}-phenyl)-methyl-carbamic acid tert-butyl ester was obtained by the method used in the preparation of 3-{2-[5-cyclohexyl-1-(3,3-dimethyl-2-oxo-butyl)-2-oxo-1,2-dihydro-3H-1,3,4-benzotriazepin-3-yl]-acetylamino}-benzoic acid benzyl ester (Example 1, step e) except that (3-amino-phenyl)-methyl-carbamic acid tert-butyl ester (Example 2, step c) was used in place of 3-amino-benzoic acid benzyl ester. ¹H NMR (CDCl₃) 8.29 (1H, s), 7.44 (3H, m), 7.29-7.11 (3H, m), 7.03-6.94 (2H, m), 4.67 (2H, m), 4.23 (2H, m), 3.23 (3H, s), 2.79 (1H, m), 2.05-1.52 (6H, m), 1.45 (9H, s), 1.37-1.23 (13H, m).

Step e. (+/−)-(3-{2-[5-Cyclohexyl-1-(3,3-dimethyl-2-oxo-butyl)-2-oxo-1,2,4,5-tetrahydro-3H-1,3,4-benzotriazepin-3-yl]-acetylamino}-phenyl)-methyl-carbamic acid tert-butyl ester was obtained by the method used in the preparation of (+/−)-3-{2-[5-cyclohexyl-1-(3,3-dimethyl-2-oxo-butyl)-2-oxo-1,2,4,5-tetrahydro-3H-1,3,4-benzotriazepin-3-yl]-acetylamino}-benzoic acid (Example 1, step f) except that (3-{2-[5-cyclohexyl-1-(3,3-dimethyl-2-oxo-butyl)-2-oxo-1,2-dihydro-3H-1,3,4-benzotriazepin-3-yl]-acetylamino}-phenyl)-methyl-carbamic acid tert-butyl ester (Example 2, step d) was used in place of 3-{2-[5-cyclohexyl-1-(3,3-dimethyl-2-oxo-butyl)-2-oxo-1,2-dihydro-3H-1,3,4-benzotriazepin-3-yl]-acetylamino}-benzoic acid benzyl ester. ¹H NMR (CDCl₃) 8.40 (1H, s), 7.66 (1H, t), 7.41 (1H, m), 7.27-7.11 (4H, m), 6.98 (2H, m), 5.35 (1H, br s), 5.14 (1H, d), 4.28 (3H, m), 3.95 (1H, d), 3.26 (3H, s), 1.73-1.50 (5H, m), 1.44-1.06 (15H, m).

Step f. A solution of the product of step e (250 mg, 0.41 mmol) in trifluoroacetic acid (3 ml) was stirred at room temperature for 1 h. The trifluoroacetic acid was evaporated under reduced pressure, the residue was partitioned between saturated NaHCO₃ (20 ml) and EtOAc (20 ml). The organic phase was separated and dried over MgSO₄. Filtration and evaporation of the solvent gave the crude product, which was purified by flash column chromatography (EtOAc-DCM (1:9)) to afford the title compound, as a colourless foam (161 mg, 78%). ¹H NMR (CDCl₃) 8.21 (1H, s), 7.27-7.01 (5H, m), 6.98 (1H, d), 6.75 (1H, dd), 6.36 (1H, dd), 5.37 (1H, d), 5.13 (1H, d), 4.35 (1H, dd), 4.27 (1H, d), 4.22 (1H, d), 4.00 (1H, d), 3.73 (1H, br s), 2.84 (3H, s), 1.74-1.68 (5H, m), 1.41-1.04 (15H, m). Found: C, 68.78, H, 7.92, N, 13.91%; C₂₉H₃₉N₅O₃ requires: C, 68.88, H, 7.77, N, 13.85%.

### Example 3

### (+/−)-2-[5-Cyclohexyl-1-(3,3-dimethyl-2-oxo-butyl)-2-oxo-1,2,4,5-tetrahydro-3H-3H-1,3,4-benzotriazepin-3-yl]-N-m-tolyl-acetamide

The title compound was obtained by the method used in the preparation of (+/−)-3-{2-[5-cyclohexyl-1-(3,3-dimethyl-2-oxo-butyl)-2-oxo-1,2,4,5-tetrahydro-3H-1,3,4-benzotriazepin-3-yl]-acetylamino}-benzoic acid (Example 1) except that m-toluidine was used instead of 3-amino-benzoic acid benzyl ester in step e. ¹H NMR (CDCl₃) 8.35 (1H, br s), 7.52 (1H, d), 7.45 (1H, s), 7.27-7.10 (4H, m), 6.98 (1H, d), 6.92 (1H, d), 5.30 (1H, br s), 5.15 (1H, d), 4.33-4.15 (3H, m), 3.95 (1H, d), 2.34 (3H, s), 1.72-1.38 (5H, m), 1.30-1.06 (15H, m). Found: C, 69.83, H, 7.87, N, 10.94%; C₂₉H₃₈N₄O₃.0.5H₂O requires: C, 69.61, H, 7.87, N, 11.19%.

### Example 4

### (+/−)-3-{2-[5-Cyclohexyl-1-(2-cyclopentyl-2-oxo-ethyl)-2-oxo-1, 2,4,5-tetrahydro-3H-1,3,4-benzotriazepin-3-yl]-acetylamino}-benzoic acid

Step a. [5-Cyclohexyl-1-(2-cyclopentyl-2-oxo-ethyl)-2-oxo-1,2-dihydro-3H-1,3,4-benzotriazepin-3-yl]-acetic acid was obtained using steps a-d of the method employed in the preparation of [5-cyclohexyl-1-(3,3-dimethyl-2-oxo-butyl)-2-oxo-1,2-dihydro-3H-1,3,4-benzotriazepin-3-yl]-acetic acid (Example 1, step d) except that 2-bromo-cyclopentyl-ethanone was used in step c instead of 1-bromo-3,3-dimethyl-butan-2-one. ¹H NMR (CDCl₃) 11.00 (1H, br s), 7.45 (2H, m), 7.25 (1H, m), 7.01 (1H, d), 4.56 (2H, d), 4.23 and 3.95 (2H, 2×d), 2.97 (1H, m), 2.81 (1H, m), 2.03-1.58 (13H, m), 1.30 (5H, m).

Step b. The title compound was obtained by the method used in the preparation of (+/−)-3-{2-[5-cyclohexyl-1-(3,3-dimethyl-2-oxo-butyl)-2-oxo-1,2,4,5-tetrahydro-3H-1,3,4-benzotriazepin-3-yl)-acetylamino}-benzoic acid (Example 1) except that [5-cyclohexyl-1-(2-cyclopentyl-2-oxo-ethyl)-2-oxo-1,2-dihydro-3H-1,3,4-benzotriazepin-3-yl]-acetic acid (Example 4, step a) was used in place of [5-cyclohexyl-1-(3,3-dimethyl-2-oxo-butyl)-2-oxo-1,2-dihydro-3H-1,3,4-benzotriazepin-3-yl]-acetic acid (Example 1, step d) in step e. ¹H NMR (CDCl₃) 8.62 (1H, s), 8.20 (2H, m), 7.84 (1H, d), 7.45 (1H, t), 7.23-7.10 (3H, m), 6.97 (1H, d), 5.31 (1H, br s), 5.04 (1H, d), 4.40-4.23 (3H, m), 3.96 (1H, d), 3.06 (1H, m), 2.01-1.65 (13H, m), 1.36-1.05 (6H, m). The compound was further characterised as the N-methyl-D-glucamine salt. Found: C, 56.69, H, 7.58, N, 8.69%; C₃₀H₃₆N₄O₅.C₇H₁₇NO₅.3.2H₂O requires: C, 56.54, H, 7.62, N, 8.91%;

### Example 5

### (+/−)-(3-{2-[5-Cyclohexyl-1-(2-cyclopentyl-2-oxo-ethyl)-2-oxo-1,2,4,5-tetrahydro-3H-1,3,4-benzotriazepin-3-yl]-acetylamino}-phenyl)-acetic acid

Step a. (3-{2-[5-Cyclohexyl-1-(2-cyclopentyl-2-oxo-ethyl)-2-oxo-1,2-dihydro-3H-1,3,4-benzotriazepin-3-yl]-acetylamino}-phenyl)-acetic acid methyl ester was obtained by the method used in the preparation of 3-(2-[5-cyclohexyl-1-(3,3-dimethyl-2-oxo-butyl)-2-oxo-1,2-dihydro-3H-1,3,4-benzotriazepin-3-yl]-acetylamino}-benzoic acid benzyl ester (Example 1, steps a-e) except that [5-cyclohexyl-1-(2-cyclopentyl-2-oxo-ethyl)-2-oxo-1,2-dihydro-3H-1,3,4-benzotriazepin-3-yl]-acetic acid (Example 4, step a) and (3-amino-phenyl)-acetic acid methyl ester were used in place of [5-cyclohexyl-1-(3,3-dimethyl-2-oxo-butyl)-2-oxo-1,2-dihydro-3H-1,3,4-benzotriazepin-3-yl]-acetic acid (Example 1, step d) and 3-amino-benzoic acid benzyl ester respectively in step e. ¹H NMR (CDCl₃) 8.28 (1H, s), 7.48 (2H, m), 7.34-7.19 (4H, m), 7.06 (1H, d), 6.98 (1H, d), 4.66 (1H, d), 4.46 (1H, d), 4.30 (1H, d), 4.17 (1H, d), 3.68 (3H, s), 3.57 (2H, s), 2.95 (1H, m), 2.79 (1H, m), 2.04-1.55 (13H, m), 1.29 (5H, m).

Step b. (3-{2-[5-Cyclohexyl-1-(2-cyclopentyl-2-oxo-ethyl)-2-oxo-1,2- dihydro-3H-1,3,4-benzotriazepin-3-yl]-acetylamino}-phenyl)-acetic acid. To a solution of the product of step a above (430 mg, 0.77 mmol) in THF-H₂O (2:1/9 ml) was added lithium hydroxide monohydrate (100 mg, 2.3 mmol) and the mixture was stirred at room temperature for 16 h. The THF was evaporated under reduced pressure, the aqueous solution was diluted with H₂O (20 ml) and acidified to pH 3 with 1N HCl. The reaction mixture was extracted with DCM (20 ml×2), the combined extracts were washed with brine (50 ml), dried over MgSO₄, filtered and the solvent was evaporated under reduced pressure to afford the product as an off-white solid (410 mg, 98%). 1H NMR (CDCl₃) 9.20 (1H, br s), 8.29 (1H, s), 7.46 (2H, m), 7.36-7.19 (4H, m), 7.06 (1H, d), 6.99 (1H, d), 4.66 (1H, d), 4.46 (1H, d), 4.32 (1H, d), 4.17 (1H, d), 3.58 (2H, s), 2.93 (1H, m), 2.78 (1H, m), 1.99-1.51 (13H, m), 1.27 (5H, m).

Step c. The title compound was obtained by the method used in the preparation of (+/−)-3-{2-[5-cyclohexyl-1-(3,3-dimethyl-2-oxo-butyl)-2-oxo-1,2,4,5-tetrahydro-3H-1,3,4-benzotriazepin-3-yl]-acetylamino}-benzoic acid (Example 1, step f) except that (3-{2-[5-cyclohexyl-1-(2-cyclopentyl-2-oxo-ethyl)-2-oxo-1,2-dihydro-3H-1,3,4-benzotriazepin-3-yl]-acetylamino}-phenyl)-acetic acid (Example 5, step b) was used in place of 3-{2-[5-cyclohexyl-1-(3,3-dimethyl-2-oxo-butyl)-2-oxo-1,2-dihydro-3H-1,3,4-benzotriazepin-3-yl]-acetylamino}-benzoic acid benzyl ester (Example 1, step e). ¹H NMR (CDCl₃) 8.41 (1H, s), 7.61 (2H, m), 7.30-7.05 (4H, m), 7.04 (1H, d), 6.95 (1H, d), 5.31 (1H, br s), 5.00 (1H, d), 4.32-4.21 (3H, m), 3.97 (1H, d), 3.62 (2H, s), 3.03 (1H, m), 2.05-1.59 (13H, m), 1.28-1.05 (6H, m). The compound was further characterised as the N-methyl-D-glucamine salt. Found: C, 57.26, H, 7.30, N, 8.61%; C₃₁H₃₈N₄O₅.C₇H₁₇NO₅.1.3H₂O requires: C, 57.25, H, 7.31, N, 8.67%.

### Example 6

### (+/−)-3-(3-(2-[5-Cyclohexyl-1-(2-cyclopenlyl-2-oxo-ethyl)-2-oxo-1,2,4,5tetrahydro-3H-1,3,4-benzotriazepin-3-yl]-acetylamino}-phenyl)-propionic acid

The title compound was obtained by the method used in the preparation of (+/−)-(3-{2-[5-cyclohexyl-1-(2-cyclopentyl-2-oxo-ethyl)-2-oxo-1,2,4,5-tetrahydro-3H-1,3,4-benzotriazepin-3-yl]-acetylamino}-phenyl)-acetic acid (Example 5), except that 3-(3-amino-phenyl)-propionic acid methyl ester was used in place of (3-amino-phenyl)-acetic acid methyl ester in step a. ¹H NMR (CDCl₃) 8.40 (1H, s), 7.53 (2H, m), 7.27-7.09 (4H, m), 6.96 (2H, d), 5.30 (1H, br s), 5.02 (1H, d), 4.28 (3H, m), 3.97 (1H, d), 3.02 (1H, m), 2.94 (2H, t), 2.68 (2H, t), 1.96-1.65 (13H, m), 1.35-1.05 (6H, m). The compound was further characterised as the N-methyl-D-glucamine salt. Found: C, 57.25, H, 7.38, N, 8.33%; C₃₂H₄₀N₄O₅.C₇H₁₇NO₅.1.0H₂O.0.7CH₂Cl₂ requires: C, 57.22, H, 7.31, N, 8.41%.

### Example 7

### (+/−)-2-[5-Cyclohexyl-1-(2-cyclopentyl-2-oxo-ethyl)-2-oxo-1,2,4,5-tetrahydro-3H-1,3,4-benzotriazepin-3-yl]-N-(3-methylamino-phenyl)-acetamide

The title compound was obtained by the method used in the preparation of (+/−)-3-{2-[5-cyclohexyl-1-(3,3-dimethyl-2-oxo-butyl)-2-oxo-1,2,4,5-tetrahydro-3H-1,3,4-benzotriazepin-3-yl]-N-(3-methylamino-phenyl)-acetamide (Example 2) except that [5-cyclohexyl-1-(2-cyclopentyl-2-oxo-ethyl)-2-oxo-1,2-dihydro-3H-1,3,4-benzotriazepin-3-yl]-acetic acid (Example 4, step a) was used in place of [5-cyclohexyl-1-(3,3-dimethyl-2-oxo-butyl)-2-oxo-1,2-dihydro-3H-1,3,4-benzotriazepin-3-yl]-acetic acid (Example 1, step d) in step d. ¹H NMR (CDCl₃) 8.22 (1H, s), 7.27-7.01 (5H, m), 7.02 (1H, d), 6.74 (1H, dd), 6.36 (1H, dd), 5.32 (1H, d), 5.00 (1H, d), 4.34 (1H, dd), 4.26 (1H, d), 4.20 (1H, d), 4.00 (1H, d), 3.03 (1H, m), 2.84 (3H, s), 1.95-1.05 (20H, m).

### Example 8

### (+/−)-3-(2-{5-Cyclohexyl-1-[2-(1-methyl-cyclohexyl)-2-oxo-ethyl]-2-oxo-1,2,4, 5-tetrahydro-3H-1,3,4-benzotriazepin-3-yl}-acetylamino)-benizoic acid

The title compound was obtained by the method used in the preparation of (+/−)-3-{2-[5-cyclohexyl-1-(3,3-dimethyl-2-oxo-butyl)-2-oxo-1,2,4,5-tetrahydro-3H-1,3,4-benzotriazepin-3-yl]-acetylamino}-benzoic acid (Example 1) except that 2-bromo-1-(1-methyl-cyclohexyl)-ethanone (prepared from 1-methyl-cyclohexane-carboxylic acid in two steps) was used in step c instead of 1-bromo-3,3-dimethyl-butan-2-one. ¹H NMR (CDCl₃) 8.68 (1H, s), 8.27 (1H, d), 8.15 (1H, s), 7.85 (1H, d), 7.45 (1H, t), 7.27-7.10 (3H, m), 6.98 (1H, d), 5.32 (1H, br s), 5.17 (1H, d), 4.46-4.27 (3H, m), 3.97 (1H, d), 2.13 (2H, m), 1.73-1.18 (22H, m). The compound was further characterised as the N-methyl-D-glucamine salt. Found: C, 58.52, H, 7.61, N, 8.38%; C₃₂H₄₀N₄O₅.C₇H₁₇NO₅.2.7H₂) requires: C, 58.25, H, 7.82, N, 8.71%.

### Example 9

### (+/−)-3-{2-[5-Cyclohexyl-1-(2-cyclohexyl-2-oxo-ethyl)-2-oxo-1,2,4,5-tetrahydro-3H-1,3,4-benzotriazepin-3-yl]-acetylamino}-benzoic acid

Step a. [5-Cyclohexyl-1-(2-cyclohexyl-2-oxo-ethyl)-2-oxo-1,2-dihydro-3H-1,3,4-benzotriazepin-3-yl]-acetic acid was obtained using steps a-d of the method employed in the preparation of [5-cyclohexyl-1-(3,3-dimethyl-2-oxo-butyl)-2-oxo-1,2-dihydro-3H -1,3,4-benzotriazepin-3-yl]-acetic acid (Example 1, step d) except that 2-bromo-cyclohexyl-ethanone was used in step c instead of 1-bromo-3,3-dimethyl-butan-2-one. ¹H NMR (CDCl₃) 7.45 (2H, m), 7.23 (1H, m), 7.01 (1H, d), 4.56 (2H, d), 4.25 (1H, d), 3.89 (1H, d), 2.82 (1H, m), 2.47 (1H, m), 2.08-1.61 (1H, m), 1.46-1.19 (9H, m).

Step b. The title compound was obtained by the method used in the preparation of (+/−)-3-{2-[5-cyclohexyl-1-(3,3-dimethyl-2-oxo-butyl)-2-oxo-1,2,4,5-tetrahydro-3H-1,3,4-benzotriazepin-3-yl]-acetylamino}-benzoic acid (Example 1) except that [5-cyclohexyl-1-(2-cyclohexyl-2-oxo-ethyl)-2-oxo-1,2-dihydro-3H-1,3,4-benzotriazepin-3-yl]-acetic acid (Example 9, step a) was used in place of [5-cyclohexyl-1-(3,3-dimethyl-2-oxo-butyl)-2-oxo-1,2-dihydro-3H-1,3,4-benzotriazepin-3-yl]-acetic acid (Example 1, step d) in step e. ¹H NMR (CDCl₃) 8.64 (1H, s), 8.32 (1H, d), 8.15 (1H, s), 7.84 (1H, d), 7.46 (1H, t), 7.27-7.10 (3H, m), 6.96 (1H, d), 5.36 (1H, br s), 5.05 (1H, d), 4.40-4.23 (3H, m), 3.95 (1H, d), 2.57 (1H, m), 2.04-1.05 (21H, m). The compound was further characterised as the N-methyl-D-glucamine salt. Found: C, 58.39, H, 7.34, N, 8.55%; C₃₁H₃₈N₄O₅.C₇H₁₇NO₅.2.5H₂O requires: C, 58.05, H, 7.68, N, 8.91%.

### Example 10

### (+/−)-(3-{2-[5-Cyclohexyl-1-(2-cyclohexyl-2-oxo-ethyl)-2-oxo-1,2,4,5-tetrahydro-3H-1,3,4-benzotriazepin-3-yl]-acetylamino}-phenyl)-acetic acid

The title compound was obtained by the method used in the preparation of (+/−)-(3-{2-[5-cyclohexyl-1-(2-cyclopentyl-2-oxo-ethyl)-2-oxo-1,2,4,5-tetrahydro-3H-1,3,4-benzotriazepin-3-yl]-acetylamino}-phenyl)-acetic acid (Example 5), except that [5-cyclohexyl-1-(2-cyclohexyl-2-oxo-ethyl)-2-oxo-1,2-dihydro-3H-1,3,4-benzotriazepin-3-yl]-acetic acid (Example 9, step a) was used in place of [5-cyclohexyl-1-(2-cyclopentyl-2-oxo-ethyl)-2-oxo-1,2-dihydro-3H-1,3,4-benzotriazepin-3-yl]-acetic acid in step a. ¹H NMR (CDCl₃) 8.41 (1H, s), 7.67 (1H, d), 7.53 (1H, s), 7.31-7.11 (4H, m), 7.04 (1H, d), 6.95 (1H, d), 5.30 (1H, br s), 5.02 (1H, d), 4.26 (3H, m), 3.97 (1H, d), 3.63 (2H, s), 2.53 (1H, m), 1.99-1.05 (21H, m). The compound was further characterised as the N-methyl-D-glucamine salt. Found: C, 60.09, H, 7.54, N, 8.44%; C₃₂H₄₀N₄O₅.C₇H₁₇NO₅.1.3H₂O requires: C, 60.27, H, 7.75, N, 8.66%.

### Example 11

### (+)-3-{2-[5-Cyclohexyl-1-(2-cyclopentyl-2-oxo-ethyl)-2-oxo-1,2,4,5-tetrahydro-3H-1,3,4-benzotriazepin-3-yl]-acetylamino}-benzoic acid

Step a. (1′R,2′S,5′R)-[5-Cyclohexyl-1-(2-cyclopentyl-2-oxo-ethyl)-2-oxo-1,2-dihydro-3H-1,3,4-benzotriazepin-3-yl]-acetic acid 2′-isopropyl-5′-methyl-cyclohexyl ester. To a solution of [5-cyclohexyl-1-(2-cyclopentyl-2-oxo-ethyl)-2-oxo-1,2-dihydro-3H-1,3,4-benzotriazepin-3-yl]-acetic acid (Example 4, step a) (1.65 g, 4.00 mmol) and (1R,2S,5R)-(−)-menthol (620 mg, 4.00 mmol) in DCM (20 ml) were added EDC (1.15 g, 6.00 mmol) and DMAP (760 mg, 6.00 mmol) and the solution was stirred under argon at room temperature for 16 h. The reaction mixture was diluted with DCM (20 ml), washed with water (20 ml), 5% KHSO₄ (20 ml), saturated NaHCO₃ (20 ml) and brine (20 ml). The organic phase was dried over MgSO₄, filtered and the solvent was evaporated under reduced pressure to afford the product as a colourless foam (2.11 g, 96%). ¹H NMR (CDCl₃) 7.38 (2H, m), 7.18 (1H, t), 6.99 (1H, d), 4.68 (1H, m), 4.64-4.16 (4H, m), 2.94 (1H, m), 2.74 (1H, m), 1.99-0.70 (36H, m).

Step b. (5R,1′S,2′S,5′R) and (5S,1′S,2′S,5′R)-[5-Cyclohexyl-1-(2-cyclopentyl-2-oxo-ethyl)-2-oxo-1,2,4,5-tetrahydro-3H1-1,3,4-benzotriazepin-3-yl]-acetic acid 2′-isopropyl-5′-methyl-cyclohexyl ester. (1′R,2′S,5′R)-[5-Cyclohexyl-1-(2-cyclopentyl-2-oxo-ethyl)-2-oxo-1,2-dihydro-3H-1,3,4-benzotriazepin-3-yl]-acetic acid 2-isopropyl-5-methyl-cyclohexyl ester (Example 11, step a) (2.10 g, 3.80 mmol) was hydrogenated according to the method employed in Example 1, step f to afford a 1:1 mixture of products (2.05 g, 97%). The diastereoisomers were separated by flash column chromatography (hexane-DCM-EtOAc (9.5:9.5:1)).

Diastereoisomer I (high R_{f}): 640 mg, 31%, α_{D}=+32.6° (c=1, DCM), ¹H NMR (CDCl₃) 7.15 (2H, m), 7.05 (1H, m), 6.91 (1H, d), 5.23 (1H, d), 4.87 (1H, d), 4.75 (1H, td), 4.36 (1H, m), 4.25 (1H, d), 4.17 (1H, d), 4.03 (1H, d), 2.97 (1H, m), 2.05-0.74 (37H, m).

Diastereoisomer II (low R_{f}): 890 mg, 43%, α_{D}=−93.2° (c=1, DCM), ¹H NMR (CDCl₃) 7.15 (2H, m), 7.05 (1H, m), 6.91 (1H, d), 5.32 (1H, d), 4.92 (1H, d), 4.74 (1H, td), 4.36 (1H, dd), 4.15 (1H, d), 4.03 (2H, m), 2.96 (1H, m), 2.03-0.77 (37H, m).

Step c. (+)-[5-Cyclohexyl-1-(2-cyclopentyl-2-oxo-ethyl)-2-oxo-1,2,4,5-tetrahydro-3H -1,3,4-benzotriazepin-3-yl]-acetic acid. To a solution of diastereoisomer I of step b above (436 mg, 0.79 mmol) in MeOH (40 ml) was added a solution of NaOH (3.2 ml of 1N solution, 3.2 mmol) and the solution was heated at reflux for 16 h. After cooling the MeOH was evaporated, the residue was diluted with water (20 ml) and the aqueous solution was extracted with Et₂O (20 ml×3). The aqueous phase was acidified to pH=3 with 1N HCl and the product was extracted with DCM (20 ml×3). The organic phase was dried over MgSO₄, filtered and the solvent was evaporated to afford the product as a white solid (311 mg, 85%). α_{D}=+116.4° (c=1.3, DCM). ¹H NMR (CDCl₃) 7.21-7.06 (3H, m), 6.93 (1H, d), 5.50 (1H, br s), 4.93 (1H, d), 4.35 (1H, d), 4.27 (1H, d), 4.10 (2H, s), 2.97 (1H, m), 1.90-1.11 (19H, m).

Step d. The title compound was obtained by the method used in the preparation of (+/−)-3-{2-[5-cyclohexyl-1-(3,3-dimethyl-2-oxo-butyl)-2-oxo-1,2,4,5-tetrahydro-3H-1,3,4-benzotriazepin-3-yl]-acetylamino}-benzoic acid (Example 1, steps e and f) except that (+)-[5-cyclohexyl-1-(2-cyclopentyl-2-oxo-ethyl)-2-oxo-1,2,4,5-tetrahydro-3H-1,3,4-benzotriazepin-3-yl]-acetic acid (Example 11, step c) was used in place of [5-cyclohexyl-1-(3,3-dimethyl-2-oxo-butyl)-2-oxo-1,2-dihydro-3H-1,3,4-benzotriazepin-3-yl]-acetic acid (Example 1, step d) in step e. α_{D}=+72.7° (c=2, MeOH). ¹H NMR (CDCl₃) 8.62 (1H, s), 8.20 (2H, m), 7.84 (1H, d), 7.45 (1H, t), 7.23-7.10 (3H, m), 6.97 (1H, d), 5.31 (1H, br s), 5.04 (1H, d), 4.40-4.23 (3H, m), 3.96 (1H, d), 3.06 (1H, m), 2.01-1.65 (13H, m), 1.36-1.05 (6H, m). The compound was further characterised as the N-methyl-D-glucamine salt. Found: C, 56.96, H, 6.81, N, 8.39%; C₃₀H₃₆N₄O₅.C₇H₁₇NO₅.0.9DCM requires: C, 56.64, H, 6.87, N, 8.72%.

### Example 12

### (−)-3-{2-[5-Cyclohexyl-1-(2-cyclopentyl-2-oxo-ethyl)-2-oxo-1,2,4,5-tetrahydro-3H-1,3,4-benzotriazepin-3-yl]-acetylamino}-benzoic acid

The title compound was obtained by the method used in Example 11, steps c and d, except that diastereoisomer II (Example 11, step b) was used in step c instead of diastereoisomer I. α_{D}=−90° (c=1.4, MeOH). ¹H NMR (CDCl₃) 8.62 (1H, s), 8.20 (2H, m), 7.84 (1H, d), 7.45 (1H, t), 7.23-7.10 (3H, m), 6.97 (1H, d), 5.31 (1H, br s), 5.04 (1H, d), 4.40-4.23 (3H, m), 3.96 (1H, d), 3.06 (1H, m), 2.01-1.65 (13H, m), 1.36-1.05 (6H, m). The compound was further characterised as the N-methyl-D-glucamine salt. Found: C, 57.77, H, 7.20, N, 8.93%; C₃₀H₃₆N₄O₅.C₇H₁₇NO₅.2H₂O requires: C, 58.09, H, 7.53, N, 9.16%.

### Example 13

### (+)-(3-{2-[5-Cyclohexyl-1-(2-cyclohexyl-2-oxo-ethyl)-2-oxo-1,2,4,5-tetrahydro-3H-1,3,4-benzotriazepin-3-yl]-acetylamino}-phenyl)-acetic acid

Step a. (5R,1′S,2′S,5′R) and (5S,1′S,2′S,5′R)-[5-Cyclohexyl-1-(2-cyclohexyl-2-oxo-ethyl)-2-oxo-1,2,4,5-tetrahydro-3H-1,3,4-benzotriazepin-3-yl]-acetic acid 2′-isopropyl-5′-methyl-cyclohexyl ester were obtained by the method of Example 11, steps a and b except that [5-cyclohexyl-1-(2-cyclohexyl-2-oxo-ethyl)-2-oxo-1,2-dihydro-3H-1,3,4-benzotriazepin-3-yl]-acetic acid (Example 9, step a) was used in place of [5-cyclohexyl-1-(2-cyclopentyl-2-oxo-ethyl)-2-oxo-1,2-dihydro-3H-1,3,4-benzotriazepin-3-yl]-acetic acid (Example 4, step a) in step a.

Diastereoisomer I (high R_{f}): α_{D}=+31.1° (c=1, DCM). ¹H NMR (CDCl₃) 7.15 (2H, m), 7.05 (1H, m), 6.91 (1H, d), 5.21 (1H, d), 4.87 (1H, d), 4.76 (1H, m), 4.36 (1H, dd), 4.20 (2H, m), 4.02 (1H, d), 2.47 (1H, m), 2.01-0.74 (39H, m).

Diastereoisomer II (low R_{f}): α_{D}=−101.7° (c=1, DCM), 1H NMR (CDCl₃) 7.15 (2H, m), 7.05 (1H, m), 6.90 (1H, d), 5.31 (1H, d), 4.93 (1H, d), 4.76 (1H, m), 4.36 (1H, dd), 4.12 (1H, d), 4.03 (2H, m), 2.45 (1H, m), 2.00-0.77 (39H, m).

Step b. (+)-[5-Cyclohexyl-1-(2-cyclohexyl-2-oxo-ethyl)-2-oxo-1,2,4,5-tetrahydro-3H -1,3,4-benzotriazepin-3-yl]-acetic acid was prepared from diastereoisomer I of step a above according to the procedure of Example 11, step c. α_{D}=+130.7° (c=1.5, DCM). ¹H NMR (CDCl₃) 7.20-7.05 (3H, m), 6.92 (1H, d), 4.92 (1H, d), 4.35 (1H, d), 4.26 (1H, d), 4.10 (2H, m), 2.47 (1H, m), 1.93-1.11 (21H, m).

Step c. (+)-(3-{2-[5-Cyclohexyl-1-(2-cyclohexyl-2-oxo-ethyl)-2-oxo-1,2,4,5-tetrahydro-3H-1,3,4-benzotriazepin-3-yl]-acetylamino}-phenyl)-acetic acid methyl ester was obtained by the method used in the preparation of 3-{2-[5-cyclohexyl-1-(3,3-dimethyl-2-oxo-butyl)-2-oxo-1,2-dihydro-3H-1,3,4-benzotriazepin-3-yl]-acetylamino}-benzoic acid benzyl ester (Example 1, step e) except that (+)-[5-cyclohexyl-1-(2-cyclohexyl-2-oxo-ethyl)-2-oxo-1,2,4,5-tetrahydro-3H-1,3,4-benzotriazepin-3-yl]-acetic acid (Example 13, step b) and (3-amino-phenyl)-acetic acid methyl ester were used in place of [5-cyclohexyl-1-(3,3-dimethyl-2-oxo-butyl)-2-oxo-1,2-dihydro-3H-1,3,4-benzotriazepin-3-yl]-acetic acid (Example 1, step d) and 3-amino-benzoic acid benzyl ester respectively. α_{D}=+172.6° (c=1.0, DCM). ¹H NMR (CDCl₃) 8.37 (1H, s), 7.60 (2H, m), 7.31-7.11 (4H, m), 7.04 (1H, d), 6.95 (1H, d), 5.32 (1H, d), 5.01 (1H, d), 4.34-4.20 (3H, m), 3.96 (1H, d), 3.70 (3H, s), 3.62 (2H, s), 2.54 (1H, m), 2.04-1.06 (21H, m).

Step d. The title compound was obtained by the method used in the preparation of (3-{2-[5-cyclohexyl-1-(2-cyclopentyl-2-oxo-ethyl)-2-oxo-1,2-dihydro-3H-1,3,4-benzotriazepin-3-yl]-acetylamino}-phenyl)-acetic acid (Example 5, step b), except that (+)-(3-{2-[5-cyclohexyl-1-(2-cyclohexyl-2-oxo-ethyl)-2-oxo-1,2,4,5-tetrahydro-3H-1,3,4-benzotriazepin-3-yl]-acetylamino}-phenyl)-acetic acid methyl ester (Example 13, step c) was used in place of (3-{2-[5-cyclohexyl-1-(2-cyclopentyl-2-oxo-ethyl)-2-oxo-1,2-dihydro-3H-1,3,4-benzotriazepin-3-yl]-acetylamino}-phenyl)-acetic acid methyl ester. α_{D}=+159° (c=1.0, DCM). ¹H NMR (CDCl₃) 8.39 (1H, s), 7.65 (1H, d), 7.55 (1H, s), 7.31-7.08 (4H, m), 7.04 (1H, d), 6.95 (1H, d), 5.30 (1H, br s), 5.01 (1H, d), 4.31-4.20 (3H, m), 3.97 (1H, d), 3.64 (2H, s), 2.54 (1H, m), 1.99-1.05 (21H, m). The compound was further characterised as the N-methyl-D-glucamine salt. Found: C, 59.58, H, 7.57, N, 8.70%; C₃₂H₄₀N₄O₅.C₇H₁₇NO₅.1.5H₂O requires: C, 59.46, H, 7.75, N, 8.89%.

### Example 14

### (−)-(3-{2-[5-Cyclohexyl-1-(2-cyclohexyl-2-oxo-ethyl)-2-oxo-1,2,4,5-tetrahydro-3H-1,3,4-benzotriazepin-3-yl]-acetylamino}-phenyl)-acetic acid

The title compound was obtained by the method used in Example 13, steps b-d, except that diastereoisomer II (Example 13, step a) was used in step b instead of diastereoisomer I. α_{D}=−147.5° (c=1.0, DCM). ¹H NMR (CDCl₃) 8.39 (1H, s), 7.65 (1H, d), 7.55 (1H, s), 7.31-7.08 (4H, m), 7.04 (11H, d), 6.95 (1H, d), 5.30 (1H, br s), 5.01 (1H, d), 4.31-4.20 (3H, m), 3.97 (1H, d), 3.64 (2H, s), 2.54 (1H, m), 1.99-1.05 (21H, m). The compound was further characterised as the N-methyl-D-glucamine salt. Found: C, 59.24, H, 7.58, N, 8.75%; C₃₂H₄₀N₄O₅.C₇H₁₇NO₅.1.8H₂O requires: C, 59.41, H, 7.74, N, 8.88%.

### Example 15

### (+/−)2-[5-Cyclohexyl-1-(2-cyclopentyl-2-oxo-ethyl)-2-oxo-1,2,4,5-tetrahydro-3H-1,3,4-benzotriazepin-3-yl]-N-[3-(2H-tetrazol-5-yl)-phenyl]-acetamide

Step a. (+/−)-2,2-Dimethyl-propionic acid 5-(3-{2-[5-cyclohexyl-1-(2-cyclopentyl-2-oxo-ethyl)-2-oxo-1,2,4,5-tetrahydro-3H-1,3,4-benzotriazepin-3-yl]-acetylamino}-phenyl)-tetrazol-2-ylmethyl ester was obtained by the method used in the preparation of (+/−)-3-{2-[5-cyclohexyl-1-(3,3-dimethyl-2-oxo-butyl)-2-oxo-1,2,4,5-tetrahydro-3H-1,3,4-benzotriazepin-3-yl]-acetylamino}-benzoic acid (Example 1), except that [5-cyclohexyl-1-2-cyclopentyl-2-oxo-ethyl)-2-oxo-1,2-dihydro-3H-1,3,4-benzotriazepin-3-yl]-acetic acid Example 4, step a) and 5-(3-amino-phenyl)-tetrazol-2-ylmethyl ester were used in place of [5-cyclohexyl-1-(3,3-dimethyl-2-oxo-butyl)-2-oxo-1,2-dihydro-3H-1,3,4-benzotriazepin-3-yl]-acetic acid (Example 1, step d) and 3-amino-benzoic acid benzyl ester respectively in step e. ¹H NMR (CDCl₃) 8.58 (1H, s), 8.33 (1H, s), 8.01 (1H, d), 7.92 (1H, d), 7.26-7.11 (3H, m), 6.97 (1H, d), 6.51 (2H, s), 5.32 (1H, br s), 5.03 (1H, d), 4.38-4.23 (3H, m), 3.99 (1H, d), 3.06 (1H, m), 1.99-1.60 (11H, m), 1.44-1.07 (17H, m).

Step b. (+/−)-2,2-Dimethyl-propionic acid 5-(3-{2-[5-cyclohexyl-1-(2-cyclopentyl-2-oxo-ethyl)-2-oxo-1,2,4,5-tetrahydro-3H-1,3,4-benzotriazepin-3-yl]-acetylamino}-phenyl)-tetrazol-2-ylmethyl ester (Example 15, step a) (360 mg, 0.53 mmol) was stirred overnight in saturated methanolic ammonia solution (30 ml) at room temperature. After concentration in vacuo, the residue was dissolved in H₂O-MeOH (10:1/22 ml) and acidified to pH 3 by the addition of 5% KHSO₄ solution. The title compound was isolated as a white solid by filtration of the reaction mixture and dried in vacuo (255 mg, 86%). ¹H NMR (CDCl₃) 9.20 (1H, s), 7.72 (1H, d), 7.60 (2H, m), 7.26-7.07 (4H, m), 6.93 (1H, d), 5.52 (1H, br s), 4.91 (1H, d), 4.37-4.25 (3H, m), 4.13 (1H, d), 2.80 (1H, m), 1.71-0.90 (19H, m). The compound was further characterised as the N-methyl-D-glucamine salt. Found: C, 57.38, H, 7.14, N, 15.37%; C₃₀H₃₆N₈O₃.C₇H₁₇NO₅.1.0H₂O.dioxane requires: C, 57.55, H, 7.31, N, 15.49%.

### Example 16

### (+/−)-2-[5-Cyclohexyl-1-(2-cyclopentyl-2-oxo-ethyl)-2-oxo-1,2,4,5-tetrahydro-3H-1,3,4-benzotriazepin-3-yl]-N-[3-(5-oxo-2,5-dihydro-[1,2,4]oxadiazol-3-yl)-phenyl]-acetamide

Step a. [5-Cyclohexyl-1-(2-cyclopentyl-2-oxo-ethyl)-2-oxo-1,2,34, 5-tetrahydro-3H-1,3,4-benzotriazepin-3-yl]-acetic acid was obtained by the method used in the preparation of (+/−)-3-{2-[5-cyclohexyl-1-(3,3-dimethyl-2-oxo-butyl)-2-oxo-1,2,4,5-tetrahydro-3H-1,3,4-benzotriazepin-3-yl]-acetylamino}-benzoic acid (Example 1, step f) except that [5-cyclohexyl-1-(2-cyclopentyl-2-oxo-ethyl)-2-oxo-1,2-dihydro-3H-1,3,4-benzotriazepin-3-yl]-acetic acid (Example 4, step a) was used in place of 3-{2-[5-cyclohexyl-1-(3,3-dimethyl-2-oxo-butyl)-2-oxo-1,2-dihydro-3H-1,3,4-benzotriazepin-3-yl]-acetylamino}-benzoic acid benzyl ester. ¹H NMR (CDCl₃) 7.20-7.05 (3H, m), 6.92 (1H, d), 4.92 (1H, d), 4.35 (1H, d), 4.19 (1H, d), 4.10 (2H, m), 2.97 (1H, m), 1.91-1.12 (19H, m).

Step b. The title compound was obtained by the method used in the preparation of 3-{2-[5-cyclohexyl-1-(3,3-dimethyl-2-oxo-butyl)-2-oxo-1,2-dihydro-3H-1,3,4-benzotriazepin-3-yl]-acetylamino}-benzoic acid benzyl ester (Example 1, step e) except that [5-cyclohexyl-1-(2-cyclopentyl-2-oxo-ethyl)-2-oxo-1,2,4,5-tetrahydro-3H-1,3,4-benzotriazepin-3-yl]-acetic acid (Example 16, step a) and 3-(3-amino-phenyl)-2H-[1,2,4]oxadiazol-5-one (WO 93/19063) were used in place of [5-cyclohexyl-1-(3,3-dimethyl-2-oxo-butyl)-2-oxo-1,2-dihydro-3H-1,3,4-benzotriazepin-3-yl]-acetic acid (Example 1, step d) and 3-amino-benzoic acid benzyl ester respectively. ¹H NMR (CDCl₃) 10.80 91H, br s), 8.90 (1H, s), 7.77 (2H, m), 7.49 (1H, d), 7.33 (1H, t), 7.27-7.08 (3H, m), 6.96 (1H, d), 5.36 (1H, d), 5.00 (1H d), 4.37-4.29 (3H, m), 4.03 (1H, d), 2.97 (1H, m), 1.79-1.07 (19H, m). The compound was further characterised as the N-methyl-D-glucamine salt. Found: C, 57.79, H, 7.02, N, 11.95%; C₃₁H₃₆N₆O₅.C₇H₁₇NO₅.1.5H₂O requires: C, 57.43, H, 7.10, N, 12.34%.

### Example 17

### (+/−)-3-{2-[5-Cycloheptyl-1-(3,3-dimethyl-2-oxo-butyl-2-oxo-1,2,4,5-tetrahydro-3H-1,3,4-benzotriazepin-3-yl]-acetylamino}-benzoic acid

The title compound is obtained using the method employed in the preparation of (+/−)-3-{2-[5-cyclohexyl-1-(3,3-dimethyl-2-oxo-butyl)-2-oxo-1,2,4,5-tetrahydro-3H-1,3,4-benzotriazepin-3-yl]-acetylamino}-benzoic acid (Example 1) except that (2-amino-phenyl)-cycloheptyl-methanone is used in step a instead of (2-amino-phenyl)-cyclohexyl-methanone.

### Example 18

### (+/−)-(3-(2-[5-Cycloheptyl-1-(3,3-dimethyl-2-oxo-butyl)-2-oxo-1,2,4,5-tetrahydro-3H-1,3,4-benzotriazepin-3-yl]-acetylamino}-phenyl)-acetic acid

The title compound is obtained using the method employed in the preparation of (+/−)-3-{2-[5-cyclohexyl-1-(3,3-dimethyl-2-oxo-butyl)-2-oxo-1,2,4,5-tetrahydro-3H-1,3,4-benzotriazepin-3-yl]-acetylamino}-benzoic acid (Example 1) except that (2-amino-phenyl)-cycloheptyl-methanone is used in step a instead of (2-amino-phenyl)-cyclohexyl-methanone and (3-amino-phenyl)-acetic acid benzyl ester replaces 3-amino-benzoic acid benzyl ester in step e.

### Example 19

### (+/−)-(3-{3-[5-Cycloheptyl-1-(3,3-dimethyl-2-oxo-butyl)-2-oxo-1,2,4,5-tetrahydro-3H-1,3,4-benzotriazepin-3-yl]-acetylamino}-phenyl)-propionic acid

The title compound is obtained using the method employed in the preparation of (+/−)-3-{2-[5-cyclohexyl-1-(3,3-dimethyl-2-oxo-butyl)-2-oxo-1,2,4,5-tetrahydro-3H-1,3,4-benzotriazepin-3-yl]-acetylamino}-benzoic acid (Example 1) except that (2-amino-phenyl)-cycloheptyl-methanone is used in step a instead of (2-amino-phenyl)-cyclohexyl-methanone and 3-(3-amino-phenyl)-propionic acid benzyl ester replaces 3-amino-benzoic acid benzyl ester in step e.

### Example 20

### (+/−)-3-{2-[5-Cycloheptyl-]-(2-cyclopentyl-2-oxo-ethyl)-2-oxo-1,2,4,5-tetrahydro-3H-1,3,4-benzotriazepin-3-yl]-acetylamino}-benzoic acid

Step a. [5-Cycloheptyl-1-(2-cyclopentyl-2-oxo-ethyl)-2-oxo-1,2-dihydro-3H-1,3,4-benzotriazepin-3-yl]-acetic acid is obtained using steps a-d of the method employed in the preparation of [5-cyclohexyl-1-(3,3-dimethyl-2-oxo-butyl)-2-oxo-1,2-dihydro-3H-1,3,4-benzotriazepin-3-yl]-acetic acid (Example 1, step d) except that (2-amino-phenyl)-cycloheptyl-methanone is used in step a instead of (2-amino-phenyl)-cyclohexyl-methanone and 2-bromo-cyclopentyl-ethanone replaces 1-bromo-3,3-dimethyl-butan-2-one in step c.

Step b. The title compound is obtained by the method used in the preparation of (+/−)-3-{2-[5-cyclohexyl-1-(3,3-dimethyl-2-oxo-butyl)-2-oxo-1,2,4,5-tetrahydro-3H-1,3,4-benzotriazepin-3-yl]-acetylamino}-benzoic acid (Example 1) except that [5-cycloheptyl-1-(2-cyclopentyl-2-oxo-ethyl)-2-oxo-1,2-dihydro-3H-1,3,4-benzotriazepin-3-yl]-acetic acid (Example 20, step a) was used in place of [5-cyclohexyl-1-(3,3-dimethyl-2-oxo-butyl)-2-oxo-1,2-dihydro-3H-1,3,4-benzotriazepin-3-yl]-acetic acid (Example 1, step d) in step e.

### Example 21

### (+/−)-(3-{2-[5-Cycloheptyl-1-(2-cyclopentyl-2-oxo-ethyl)-2-oxo-1,2,4,5-tetrahydro-3H-1,3,4-benzotriazepin-3-yl]-acetylamino}-phenyl)-acetic acid

The title compound is obtained using the method employed in the preparation of (+/−)-3-{2-[5-cyclohexyl-1-(3,3-dimethyl-2-oxo-butyl)-2-oxo-1,2,4,5-tetrahydro-3H-1,3,4-benzotriazepin-3-yl]-acetylamino}-benzoic acid (Example 1) except that [5-cycloheptyl-1-(2-cyclopentyl-2-oxo-ethyl)-2-oxo-1,2-dihydro-3H-1,3,4-benzotriazepin-3-yl]-acetic acid (Example 20, step a) and (3-amino-phenyl)-acetic acid benzyl ester are used in place of [5-cyclohexyl-1-(3,3-dimethyl-2-oxo-butyl)-2-oxo-1,2-dihydro-3H-1,3,4-benzotriazepin-3-yl]-acetic acid (Example 1, step d) and 3-amino-benzoic acid benzyl ester respectively in step e.

### Example 22

### (+/−)-(3-{3-[5-Cycloheptyl-1-(2-cyclopentyl-2-oxo-ethyl)-2-oxo-1,2,4,5-tetrahydro-3H-1,3,4-benzotriazepin-3-yl]-acetylamino}-phenyl)-propionic acid

The title compound is obtained using-the method employed in the preparation of (+/−)-3-{2-[5-cyclohexyl-1-(3,3-dimethyl-2-oxo-butyl)-2-oxo-1,2,4,5-tetrahydro-3H-1,3,4-benzotriazepin-3-yl]-acetylamino}-benzoic acid (Example 1) except that [5-cycloheptyl-1-(2-cyclopentyl-2-oxo-ethyl)-2-oxo-1,2-dihydro-3H-1,3,4-benzotriazepin-3-yl]-acetic acid (Example 20, step a) and (3-amino-phenyl)-propionic acid benzyl ester are used in place of [5-cyclohexyl-1-(3,3-dimethyl-2-oxo-butyl)-2-oxo-1,2-dihydro-3H-1,3,4-benzotriazepin-3-yl]-acetic acid (Example 1, step d) and 3-amino-benzoic acid benzyl ester respectively in step e.

### Example 23

### (+/−)2-[5-Cycloheptyl-1-(2-cyclopentyl-2-oxo-ethyl)-2-oxo-1,2,4,5-tetrahydro-3H-1,3,4-benzotriazepin-3-yl]-N-[3-(2H-tetrazol-5-yl)-phenyl]-acetamide

Step a. (+/−)-2,2-Dimethyl-propionic acid 5-(3-{2-[5-cycloheptyl-1-(2-cyclopentyl-2-oxo-ethyl)-2-oxo-1,2,4,5-tetrahydro-3H-1,3,4-benzotriazepin-3-yl]-acetylamino}-phenyl)-tetrazol-2-ylmethyl ester is obtained by the method used in the preparation of (+/−)-3-{2-[5-cyclohexyl-1-(3,3-dimethyl-2-oxo-butyl)-2-oxo-1,2,4,5-tetrahydro-3H-1,3,4-benzotriazepin-3-yl]-acetylamino}-benzoic acid (Example 1) except that [5-cycloheptyl-1-(2-cyclopentyl-2-oxo-ethyl)-2-oxo-1,2-dihydro-3H-1,3,4-benzotriazepin-3-yl]-acetic acid (Example 20, step a) and 5-(3-amino-phenyl)-tetrazol-2-ylmethyl ester are used in place of [5-cyclohexyl-1-(3,3-dimethyl-2-oxo-butyl)-2-oxo-1,2-dihydro-3H-1,3,4-benzotriazepin-3-yl]-acetic acid (Example 1, step d) and 3-amino-benzoic acid benzyl ester respectively in step e.

Step b. The title compound is obtained by the method employed in the preparation of (+1-)-2-[5-cyclohexyl-1-(2-cyclopentyl-2-oxo-ethyl)-2-oxo-1,2,4,5-tetrahydro-3H-1,3,4-benzotriazepin-3-yl]-N-[3-(2H-tetrazol-5-yl)-phenyl]-acetamide (Example 15, step b) except that (+/−)-2,2-dimethyl-propionic acid 5-(3-{2-[5-cycloheptyl-1-(2-cyclopentyl-2-oxo-ethyl)-2-oxo-1,2,4,5-terahydro-3H-1,3,4-benzotriazepin-3-yl]-acetylamino}-phenyl)-tetrazol-2-ylmethyl ester (Example 23, step a) is used instead of (+/−)-2,2-dimethyl-propionic acid 5-(3-{2-[5-cyclohexyl-1-(2-cyclopentyl-2-oxo-ethyl)-2-oxo-1,2,4,5-terahydro-3H-1,3,4-benzotriazepin-3-yl]-acetylamino}-phenyl)-tetrazol-2-ylmethyl ester (Example 15, step a).

### Example 24

### (+/−)-2-[5-Cycloheptyl-1-(2-cyclopentyl-2-oxo-ethyl)-2-oxo-1,2-dihydro-3H-1,3,4-benzotriazepin-3-yl]-N-[3-(5-oxo-2,5-dihydro-[1,2,4]oxadiazol-3-yl)-phenyl]-acetamide

Step a. [5-Cycloheptyl-1-(2-cyclopentyl-2-oxo-ethyl)-2-oxo-1,2-dihydro-3H-1,3,4-benzotriazepin-3-yl]-acetic acid is obtained using steps a-d of the method employed in the preparation of [5-cyclohexyl-1-(3,3-dimethyl-2-oxo-butyl)-2-oxo-1,2-dihydro-3H-1,3,4-benzotriazepin-3-yl]-acetic acid (Example 1, step d) except that (2-amino-phenyl)-cycloheptyl-methanone is used in step a instead of (2-amino-phenyl)-cyclohexyl-methanone and 2-bromo-cyclopentyl-ethanone replaces 1-bromo-3,3-dimethyl-butan-2-one in step c.

Step b. (+/−)-[5-Cycloheptyl-1-(2-cyclopentyl-2-oxo-ethyl)-2-oxo-1,2,4,5-tetrahydro-3H -1,3,4-benzotriazepin-3-yl]-acetic acid was obtained by the method used in the preparation of (+/−)-3-{2-[5-cyclohexyl-1-(3,3-dimethyl-2-oxo-butyl)-2-oxo-1,2,4,5-tetrahydro-3H-1,3,4-benzotriazepin-3-yl]-acetylamino}-benzoic acid (Example 1, step f) except that [5-cycloheptyl-1-(2-cyclopentyl-2-oxo-ethyl)-2-oxo-1,2-dihydro-3H-1,3,4-benzotriazepin-3-yl]-acetic acid (Example 24 step a) was used in place of 3-{2-[5-cyclohexyl-1-(3,3-dimethyl-2-oxo-butyl)-2-oxo-1,2-dihydro-3H-1,3,4-benzotriazepin-3-yl]-acetylamino}-benzoic acid benzyl ester.

Step c. The title compound is obtained by the method used in the preparation of 3-{2-[5-cyclohexyl-1-(3,3-dimethyl-2-oxo-butyl)-2-oxo-1,2-dihydro-3H-1,3,4-benzotriazepin-3-yl]-acetylamino}-benzoic acid benzyl ester (Example 1, step e) except that (+/−)-[5-cycloheptyl-1-(2-cyclopentyl-2-oxo-ethyl)-2-oxo-1,2,4,5-tetrahydro-3H-1,3,4-benzotriazepin-3-yl]-acetic acid (Example 24, step b) and 3-(3-amino-phenyl)-2H-[1,2,4]oxadiazol-5-one (WO 93/19063) are used in place of [5-cyclohexyl-1-(3,3-dimethyl-2-oxo-butyl)-2-oxo-1,2-dihydro-3H-1,3,4-benzotriazepin-3-yl]-acetic acid (Example 1, step d) and 3-amino-benzoic acid benzyl ester respectively.

CCK₂ Antagonist Activity

A number of compounds were tested at human gastrin (CCK₂) receptors which have been cloned into an NIH3T3 cell line as follows:

Step a: Subcloning of IMAGE Clone Encoding the Human CCK₂R into a Mammalian Expression Vector

I.M.A.G.E. (Integrated Molecular Analysis of Genomes and their Expression) clone number 3504160 (Lennon et al. Genomics 33, 151-152 (1996)) was purchased from the HGMP (Human Genome Mapping Project, Cambridge). The cDNA encoding the mRNA for the human CCK₂R, corresponding to accession number BC000740, was present in vector pOTB7 in host cell DH10B. The cells, initially streaked on to LB-Agar plates containing 20 μg/ml chloramphenicol, were then grown in LB containing 20 μg/ml chloramphenicol with shaking at 37° C. according to standard techniques (Current Protocols in Molecular Biology, Wiley). DNA was prepared using the QIAGEN® EndoFree™ plasmid Maxi kit (Qiagen Ltd.) according to the manufacturer's protocol. The DNA was then amplified by PCR (polymerase chain reaction) from the start codon to the stop codon using primers containing restriction sites, Eco RI and XbaI respectively, to facilitate uni-directional cloning. The start codon primer also contained a Kozak consensus site (Kozak M, Nucleic Acids Res. 1984 Jan. 25;12(2):857-72) for optimal initiation of translation. Primers 1 and 2 (see Table 1) were synthesised to HPLC grade by Invitrogen. The PCR was performed in 20 mM Tris-HCl (pH 8.4), 50 mM KCl containing 2 mM MgCl₂, 0.2 mM dNTP (Invitrogen) and 0.1 μM of each primer. A hot start PCR was used: the samples were denatured for 2 min at 95° C., cooled to 75° C., then 1U Taq Polymerase (Invitrogen) was added and the reactions were cycled 30 times at 95° C. for 1 min, 60° C. for 30 sec and 72° C. for 3 min. The samples were cooled to 4° C., after a final extension at 72° C. for 5 min.

The PCR product was purified using the QIAGEN® MinElute™ PCR purification kit, according to the manufacturer's instructions. The PCR product and a mammalian expression vector were digested using both Eco RI (Promega Corp.) and XbaI (Promega Corp.) in 1×Buffer H (90 mM Tris-HCl, 10 mM MgCl₂, 50 mM NaCl, pH 7.5) (Promega Corp.) and 0.1 μg/ml BSA. The digested DNA bands of the correct size, analysed by ethidium bromide stained agarose/TBE gels, were excised and purified using QIAGEN® MinElute™ gel extraction kit, according to the manufacturer's instructions. The PCR product was then ligated into the vector using the Lightning™ DNA Ligation kit (Bioline) and transformed into Escherichia coli, strain XL1-Blue cells (Stratagene) according to the manufacturer's instructions.

Colonies were selected and screened using restriction digestion of DNA prepared from small-scale cultures (5 ml) using QIAGEN® plasmid Mini-prep columns. One positive clone was cultured on a larger scale (100 ml) using standard techniques (Current Protocols in Molecular Biology, Wiley) and DNA was prepared using QIAGEN® plasmid Maxi-prep columns. The DNA was then custom sequenced by MWG Biotech AG using primers 3, 4 and 5 (Table A). The sequence contained the correct sequence of primers 1 and 2 and the sequence of the coding region exactly matched that of accession number BC000740.

Step b: Generation of Stable Cell Line

NIH3T3 cells from (ECACC) were cultured in Dulbecco's modified Eagle's medium (DMEM) (Invitrogen), containing 2 mM Glutamax I (Invitrogen), 10% heat inactivated newborn calf serum (Invitrogen). Cells (4×10⁵) were seeded into 35 mm×10 mm dishes (Corning) and transfected using the Transfast™ reagent (Promega Corp.) according to the manufacturers instructions using 10 μg of the hCCK₂R plasmid DNA at a ratio of 1:1 (DNA:Transfast™ reagent). Untransfected cells and cells transfected with vector only were also prepared as controls. After 48 h the cells were trypsinised using standard techniques (Culture of Animal Cells, A Manual of Basic Techniques 4^{th} Ed, R. Ian Freshney) and dilutions were plated on 35 mm×10 mm dishes in media containing 800 μg/ml G-418 (Invitrogen). Cells were selected for 2 weeks until individual, separate colonies appeared. In the untransfected cells all cells had died after this time, confirming adequate selection. Using cloning rings and trypsinisation, according to standard techniques (Culture of Animal Cells, A Manual of Basic Techniques 4^{th} Ed, R. Ian Freshney), individual colonies were picked from plates containing cells transfected with the vector only and cells transfected with the hCCK₂R plasmid construct. The cells were expanded and analysed by radioligand binding analysis (see below).

TABLE A
Primers used in the cloning and sequence analysis of human CCK_{2S}R

| | | | Gene | |
|---|---|---|---|---|
| Primer | Primer | Sequence | specific | Restriction |
| Name | Orientation | (5′-3′) | or vector | Site |
| 1 | Forward | TCTGAATTCGCCGCCATGGAGCTGCTA | GENE | Eco RI |
| 2 | Reverse | GTATCTAGACTCAGCCAGGGCCCAGTG | GENE | Xba I |
| 3 (T7) | Forward | TAATACGACTCACTATAGG | VECTOR | — |
| 4 (T3) | Reverse | ATTAACCCTCACTAAAGGG | VECTOR | — |
| 5 | Forward | TGTCCGGACTACTCATGGTG | GENE | — |

Restrictions site in bold. Start codon underlined. Stop codon italic and underlined.

Step c: Clonal Selection

Stable clones expressing hCCK₂R were screened for their ability to specifically bind [¹²⁵I]-BH-CCK-8S in tissue concentration curve studies (0.3×10⁴-1×10⁶ cells per tube) using the assay conditions described below. Of those tested, clone 7 gave the highest amount of specifically bound and % specific bound label whilst also meeting the criteria that the amount of total bound label did not exceed 10% of the total added radio label (e.g. 4.2%). In addition there was a direct linear correlation between the amount of specific bound label and the cell concentration up to and including 2.5×10⁵ cells per ml. Based on the above, this clone was chosen for expansion and fall binding characterisation.

Step d: Membrane Preparation

Cultured clone 7 cells were stored as frozen pellets at −70° C. until required. Cell pellets were thawed in CCK₂ assay buffer ((mM): 10 Hepes; 130 NaCl; 5 MgCl₂; 4.7 KCl; 1 EGTA (pH7.2 at 21° C.) with 0.125 g Bacitracin added to each litre), and homogenised using a Polytron (4×1 s). The resulting membrane preparation was centrifuged at 39,800 g for 15 min at 4° C. Each cell pellet was re-suspended in fresh buffer and re-centriflged as above. The final pellet was re-suspended by homogenisation (Teflon-in-glass), to the appropriate membrane concentration.

Step e: Incubation Conditions

For saturation and competition studies, the cell membranes prepared as in step d (3×10⁴ cells per 400 μl) were incubated for 150 min at 21° C. in a final volume of 0.5 ml with CCK₂ assay buffer containing [¹²⁵I]-BH-CCK-8S (50 μl; 200 pM). Total and non-specific binding of [¹²⁵I]-BH-CCK-8S were defined, respectively using 50 μl of buffer and 50 μl of 10 μM YM022. The assays were terminated by rapid filtration through pre-soaked Whatman GF/B filters which were washed (3×3 ml) with ice-cold 50 mM Tris HCl (pH7.4 @ 4° C.). Filters were transferred to plastic gamma counter vials and bound radioactivity determined by counting (1 min) in a Clini-gamma counter.

Step f: Saturation Analysis

The binding of [¹²⁵I]-BH-CCK-8S to the hCCK₂ receptor isoform was saturable. Scatchard plots appeared linear and the mean slope of the corresponding Hill plots was not significantly different from unity (0.97±0.08; n=4). The equilibrium dissociation constant (pK_{D}) and Bmax values were 10.75±0.08 and 1.1±0.3 fmol per 1×10⁵ cells, respectively (n=4±s.e. mean). Saturation data were analysed using the curve-fitting programmes, Radlig and Ligand.

Step g: Competition Studies

A number of compounds of the invention as well as reference compounds were tested for their ability to displace [¹²⁵I]-BH-CCK-8S from the receptors prepared as above. Briefly, dilution and addition of test compounds, radioligand and cell membranes were performed using a Beckman Biomek 2000. The ability of compounds to inhibit the specific binding of to hCCK₂ receptors was determined in triplicate over a range of concentrations at half-log intervals. Total and non-specific binding was determined for each compound. Each compound was tested in a minimum of three experiments. Competition data were fitted to the Hill equation using Graph-pad Prism software to obtain estimates of the IC₅₀ (mid-point location parameter) and n_{H} (mid-point slope parameter). Dissociation constants (K_{I}) were determined using the Cheng & Prusoff equation (1973) to correct for the receptor occupancy by the radioligand. All compounds were dissolved in DMF to give a stock concentration of either 1 or 10 mM and subsequent dilutions were made in assay buffer. The pK_{I} (and standard error of the mean) for representative examples together with a number of reference compounds are shown in the table below. All Hill slopes were not significantly different from unity.

The results obtained at CCK₂ receptors are set out in the following Table.

| Example No | Structure | CCK₂ (pK_{I}) |
|---|---|---|
| 1 | 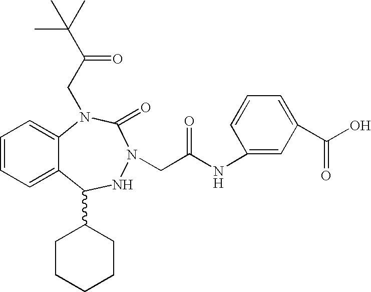 | 7.75 ± 0.13 |
| 2 | 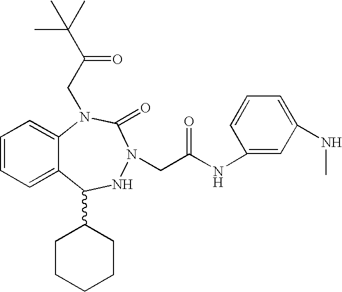 | 7.79 ± 0.09 |
| 3 | 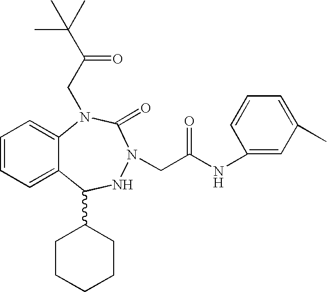 | 7.35 ± 0.13 |
| 4 | 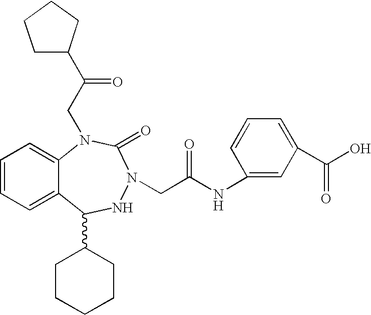 | 8.83 ± 0.03 |
| 5 | 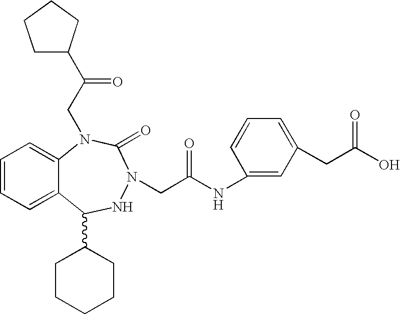 | 7.10 ± 0.33 |
| 6 | 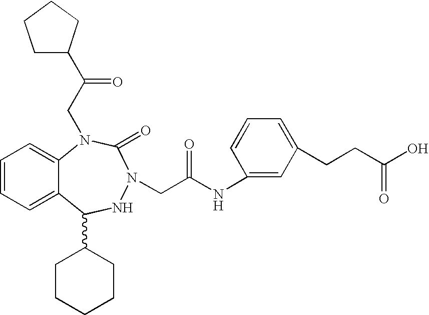 | 9.12 ± 0.02 |
| 7 | 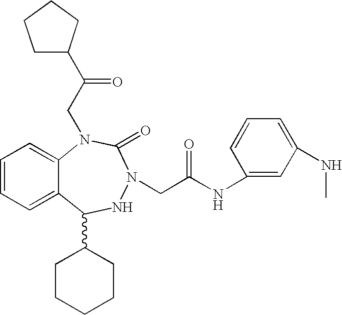 | 8.58 ± 0.19 |
| 8 | 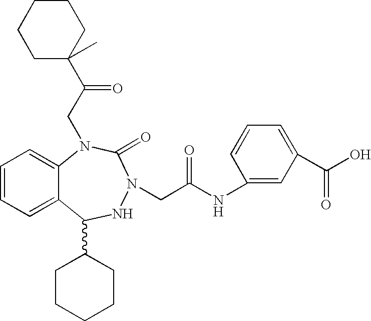 | 8.11 ± 0.14 |
| 9 | 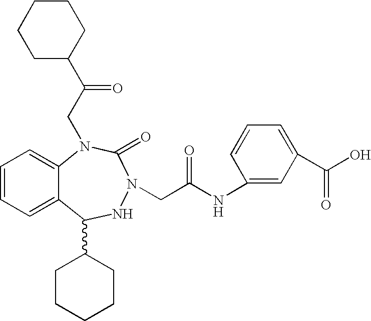 | 8.65 ± 0.13 |
| 10 | 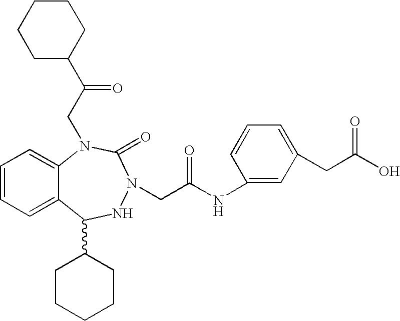 | 9.44 ± 0.14 |
| 11 | 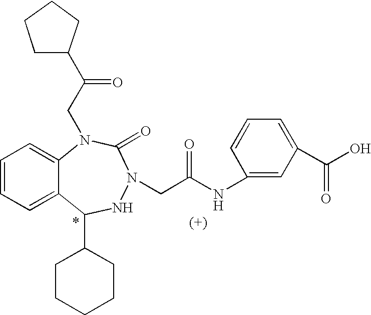 | 7.9 |
| 12 | 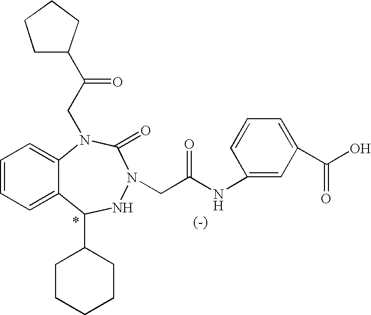 | 8.8 |
| 13 | 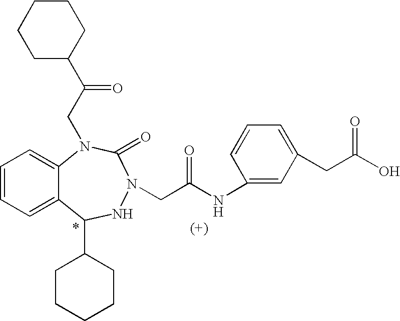 | 8.4 |
| 14 | 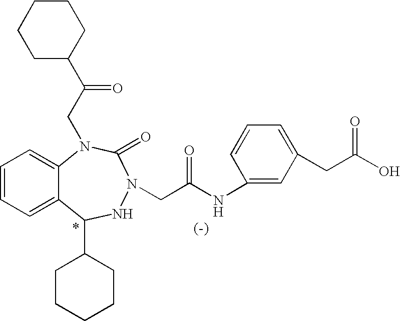 | 10.0 |
| 15 | 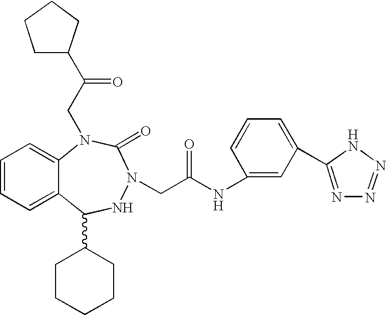 | 9.3 |
| 16 | 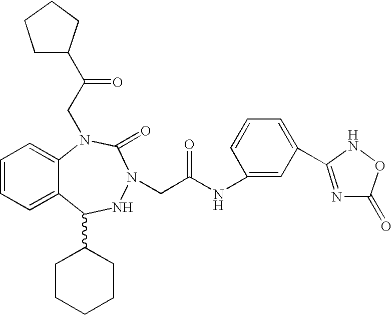 | 10.1 |
| 17 | 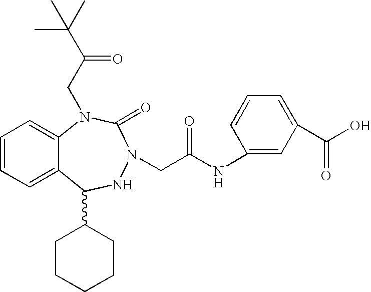 | |
| 18 | 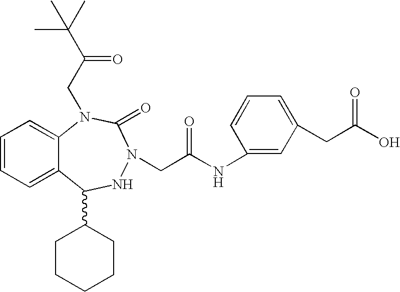 | |
| 19 | 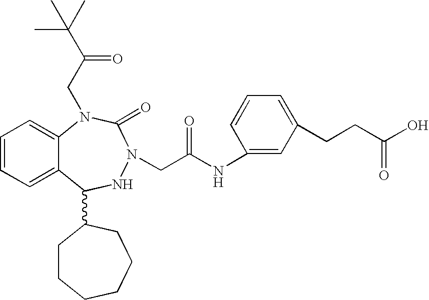 | |
| 20 | 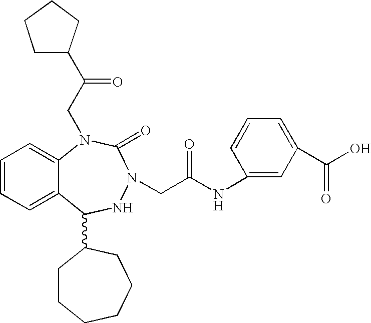 | |
| 21 | 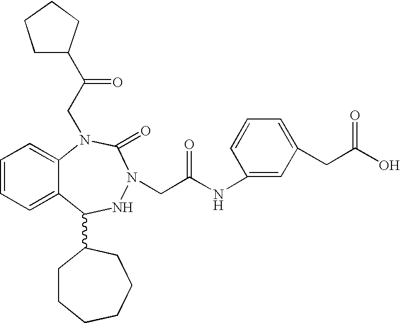 | |
| 22 | 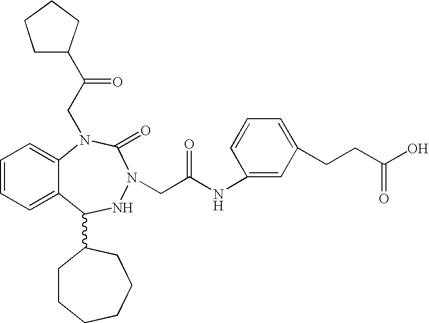 | |
| 23 | 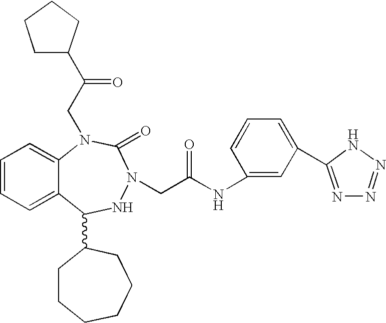 | |
| 24 | 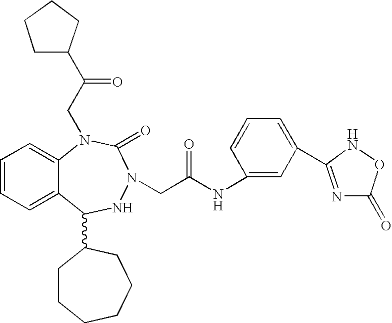 | |

Compounds of certain examples were tested in a CCK₁ receptor binding assay. All examples tested were found to have a CCK₁ pKᵢ not exceeding 5.5.

It is found that the compositions and products of the present invention comprising a compound of formula (I) and a proton pump inhibitor reduce hyperplasia, associated with administration of proton pump inhibitors. This was measured according to the following experimental protocol.

Animals and Treatment:

40 male SPF Wistar rats (200 g) were divided into 4 treatment groups and 2 strata. The treatment of the 20 rats in the second stratum started 2 weeks after the treatment of the first stratum. The design of the study was completely randomised double blind with individual blinding; all rats were placed in a separate cage. Animals had continuous access to water and food.

Animals were treated once daily during 14 days:
Control group: 1 ml gastrin test drug vehicle+1 ml p.o.(gavage) 0.25% Methocel (Dow Corning)
PPI group: 1 ml gastrin test drug vehicle+1 ml p.o.(gavage) 25 mg/kg Rabeprazole in 0.25% Methocel.
GRA group: 1 ml gastrin test drug+1 ml p.o. (gavage) 0.25% Methocel
GRA-PPI group: 1 ml gastrin test drug+1 ml p.o.(gavage) 25 mg/kg Rabeprazole in 0.25% Methocel.

Gastrin test drug made up to an appropriate dose in physiologically compatible solvent.

Preparation of Tissue:

After removal of the fundus, the stomach were rinsed with phosphate buffered saline prior to fixation with 4% formalin in Millonig buffer. After 4 hours immersion in fixative solutions at room temperature, tissue was rinsed in phosphate buffered saline (PBS), dehydrated and embedded in paraffin using the Leitz paraffin embedding station (Leitz TP 1050; Germany) dehydration module and paraffin embedding module (Leitz EG 1160; Germany).

Cross sections (3 μm thick) of the oxyntic part of the stomach were made at 3 levels, each separated by a distance of 400 μm.

Immunostaining

The following indirect immunofluorescence labeling method was used:
removal of paraffin and rehydratation of the sections followed by a blocking step
primary antibodies: polyclonal guinea pig anti-histidine decarboxylase, 1/2000 (from Euro-Diagnostica) and monoclonal mouse anti PCNA 1/2500 (Clone PC10 from Sigma). All antibodies were diluted in a 0.2% BSA solution. Sections were incubated overnight at 4° C. and then washed with a BSA solution.
secondary antibodies: goat anti guinea pig coupled to CY5, 1/500 (from Jackson Laboratories) and goat anti-mouse coupled to Cy3, 1/250 (from Jackson Laboratories); incubation for 4 hours at 37° C. After rinsing with BSA and PBS solutions, sections were mounted with slowfade (Molecular Probes Europe BV), and stored at 4° C.

Imaging

Fluorescence labelling was observed with an epifluorescence microscope or a Zeiss LSM510 (Carl Zeiss Jena GmbH) confocal microscope.

By using CY5- and CY3-coupled antibodies, the high autofluorescence properties of the oxyntic mucosa were circumvented when sections are illuminated by a 488 nm (FITC channel) light source. Negative controls, by omitting the primary antibodies, and an isotype control staining for PCNA showed complete absence of staining. The specific labelling of PCNA was checked using double staining with TOPRO-3® (Molecular Probes Europe BV), a nuclear stain. Only in the most luminal located epithelial cells, non-specific cytoplasmic labelling was present. In the glandular part of the mucosa, non-specific PCNA-staining was absent.

For determination of the labelling index of ECL cells, at least 80 confocal images per rat were taken from the 3 slides at the 3 different levels. The ratio of double labelled cells (HDC+PCNA) and all HDC labelled cells yielded the labelling index of ECL cells.

Proliferation activity of ECL cells in the PPI group is expected to be increased compared with sham, GRA and GRA-PPI groups (Eissele, R., Patberg, H., Koop, H., Krack, W., Lorenz, W., McKnight, A. T., and Arnold, R. Effect of gastrin receptor blockade on endrocine cells in rats during achlorhydria. Gastroenterology, 103, 1596-1601, 1992). Increased proliferation by PPI will be completely blocked by GRA.

1. A compound of formula (I): 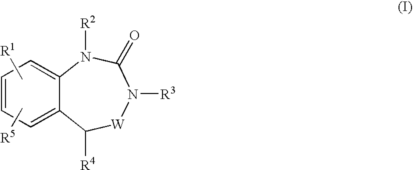
wherein:
W is NZ or NO;
R¹ and R⁵ are independently H, C₁ to C₆ alkyl, (C₁ to C₆ alkyl)oxy, thio, (C₁ to C₆ alkyl)thio, carboxy, carboxy(C₁ to C₆ alkyl), formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, hydroxy(C₁ to C₆ alkyl), amino, (C₁ to C₆ alkyl)amino, di(C₁ to C₆ alkyl)amino, aminocarbonyl, halo, halo(C₁ to C₆ alkyl), aminosulfonyl, (C₁ to C₆ alkyl)sulfonylamino, (C₁ to C₆ alkyl)aminocarbonyl, di(C₁ to C₆ alkyl)aminocarbonyl, [N-Z](C₁ to C₆ alkyl)carbonylamino, formyloxy, formamido, (C₁ to C₆ alkyl)aminosulfonyl, di(C₁ to C₆ alkyl)aminosulfonyl, [N-Z](C₁ to C₆ alkyl)sulfonylamino or cyano; or R¹ and R⁵ together form a methylenedioxy group;
R² is selected from H or an optionally substituted C₁ to C₁₈ hydrocarbyl group wherein up to three C atoms may optionally be replaced by N, O and/or S atoms;
R³ is —(CR¹¹R¹²)ₘ—X—(CR¹³R¹⁴)ₚ—R⁹;
m is 0, 1, 2, 3 or 4;
p is 0, 1 or 2;
X is a bond, —CR¹⁵═CR¹⁶—, —C≡C—, C(O)NH, NHC(O), C(O)NMe, NMeC(O), C(O)O, NHC(O)NH, NHC(O)O, OC(O)NH, NH, O, CO, SO₂, SO₂NH, C(O)NHNH, 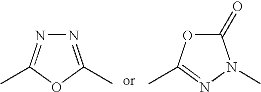
R⁹ is H; C₁ to C₆ alkyl; or phenyl, naphthyl, pyridyl benzimidazolyl, indazolyl, quinolinyl, isoquinolinyl, tetrahydroisoquinolinyl, indolinyl, isoindolinyl, indolyl, isoindolyl or 2-pyridonyl, all optionally substituted with 1, 2, or 3 groups independently selected from
-L-Q
wherein:
L is a bond, or a group of the formula —(CR¹⁷R¹⁸)ᵥ—Y—(CR¹⁷R¹⁸)_{w}, wherein v and w are independently, 0, 1, 2 or 3, and Y is a bond, —CR^{15═CR}¹⁶—, phenyl, furanyl, thiophenyl, pyrrolyl, thiazolyl, imidazolyl, oxazolyl, isoxazolyl, pyrazolyl, isoxazolonyl, piperazinyl, piperidinyl, morpholinyl, pyrrolidinyl, isothiazolyl, triazolyl, oxadiazolyl, thiadiazolyl, pyridyl or pyridazyl; and
Q is H, (C₁ to C₆ alkyl)oxy, [N-Z](C₁ to C₆ alkyl)oxy(C₁ to C₆ alkyl)amino, thio, (C₁ to C₆ alkyl)thio, carboxy(C₁ to C₆ alkyl)thio, carboxy, carboxy(C₁ to C₆ alkyl), carboxy(C₁ to C₆ alkenyl), [N-Z]carboxy(C₁ to C₆ alkyl)amino, carboxy(C₁ to C₆ alkyl)oxy, formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, amino, [N-Z](C₁ to C₆ alkyl)amino, aminocarbonyl, (C₁ to C₆ alkyl)aminocarbonyl, di(C₁ to C₆ alkyl)aminocarbonyl, [N-Z](C₁ to C₆ alkyl)carbonylamino, C₅ to C₈ cycloalkyl, [N-Z](C₁ to C₆ alkyl)carbonyl(C₁ to C₆ alkyl)amino, halo, halo(C₁ to C₆ alkyl), sulfamoyl, [N-Z](C₁ to C₆ alkyl)sulfonylamino, (C₁ to C₆ alkyl)sulfonylaminocarbonyl, carboxy(C₁ to C₆ alkyl)sulfonyl, carboxy(C₁ to C₆ alkyl)sulfinyl, tetrazolyl, [N-Z]tetrazolylamino, cyano, amidino, amidinothio, SO₃H, formyloxy, formamido, C₃ to C₈ cycloalkyl, (C₁ to C₆ alkyl)sulphamoyl, di(C₁ to C₆ alkyl)sulphamoyl, (C₁ to C₆ alkyl)carbonylaminosulfonyl, 5-oxo-2,5-dihydro[1,2,4]oxadiazolyl, carboxy(C₁ to C₆ alkyl)carbonylamino, tetrazolyl(C₁ to C₆ alkyl)thio, [N-Z]tetrazolyl(C₁ to C₆ alkyl)amino, 5-oxo-2,5-dihydro[1,2,4]thiadiazolyl, 5-oxo-1,2-dihydro[1,2,4]triazolyl, [N-Z](C₁ to C₆ alkyl)amino(C₁ to C₆ alkyl)amino, or a group of the formula 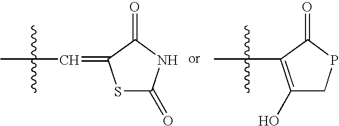
wherein P is O, S or NR¹⁹;
Z is H, C₁ to C₆ alkyl, t-butoxycarbonyl, acetyl, benzoyl or benzyl;
R⁴ is an optionally substituted C₁ to C₁₈ hydrocarbyl group wherein up to three C atoms may optionally be replaced by N, O and/or S atoms;
R¹¹, R¹², R¹³, R¹⁴, R¹⁵, R¹⁷, R¹⁸ and R¹⁹ are independently H or C₁ to C₃ alkyl; and
R¹⁶ is H, C₁ to C₃ alkyl, or acetylamino;
or a pharmaceutically acceptable salt thereof
with the proviso that when R¹ is chloro, R² is H, R⁴ is phenyl and R⁵ is H; R³ may not be H, methyl or 2-hydroxyethyl.

2. The compound according to claim 1 wherein W is NH.

3. The compound according claim 1 wherein R¹ and R⁵ are both H.

4. The compound according to claim 1 wherein R² is:
—(CH₂)ₛ—C(R⁶R⁷)ₙ—(CH₂)ₜ—R⁸
wherein:
R⁶ and R⁷ are independently selected from H, C₁ to C₆ alkyl or OH; or R⁶ and R⁷ together represent an ═O group;
n is 0 or 1;
s is 0, 1, 2 or 3;
t is 0, 1, 2 or 3; and
R⁸ is selected from H, OH, C₁ to C₁₂ alkyl, (C₁ to C₁₂ alkyl)oxy, C₃ to C₁₂ cycloalkyl, phenyl, naphthyl, pyridyl, pyrrolyl, imidazolyl, pyrazolyl, pyridazinyl, pyrimidinyl, triazolyl, furanyl, thienyl, furazanyl, oxazolyl, isoxazolyl, thiazolyl, thiazinyl, indolyl, indolinyl, isoindolyl, isoindolinyl, isoquinolinyl, quinolinyl, benzofuranyl, benzothienyl, piperazinyl, piperidinyl, pyrrolidinyl, pyrrolinyl, dihydropyranyl, tetrahydropyranyl, pyranyl, tetrahydrofuranyl, morpholinyl, thiazolidinyl, thiomorpholinyl or thioxanyl (all optionally substituted with 1, 2 or 3 groups independently selected from C₁ to C₆ alkyl, (C₁ to C₆ alkyl)oxy, thio, (C₁ to C₆ alkyl)thio, carboxy, carboxy(C₁ to C₆ alkyl), formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, hydroxy(C₁ to C₆ alkyl), amino, (C₁ to C₆ alkyl)amino, di(C₁ to C₆ alkyl)amino, aminocarbonyl, halo, halo(C₁ to C₆ alkyl), aminosulfonyl, (C₁ to C₆ alkyl)sulfonylamino or cyano).

5. The compound according to claim 4 wherein —C(R⁶R⁷)ₙ— is —C(O)—.

6. The compound according to clam 4 wherein s is 1.

7. The compound according to claim 4 wherein, t is 0 and R⁸ is a C₃ to C₁₂ cycloalkyl group (optionally substituted with a methyl group) or a branched C₃ to C₁₂ alkyl group.

8. The compound according to claim 4 wherein s is 1, t is 0 and R⁸ is a C₃ to C₁₂ cycloalkyl group or a branched C₃ to C₁₂ alkyl or group.

9. The compound according to claim 4 wherein R⁸ is a t-butyl, cyclohexyl, 1-methylcyclohexyl, 1-methylcyclopentyl or cyclopentyl group.

10. The compound according to claim 4 wherein R⁸ is a t-butyl or cyclopentyl group.

11. The compound according to claim 1 wherein m is 1, R¹¹ is H and R¹² is H

12. The compound according to claim 1 wherein p is 0.

13. The compound according to claim 1 wherein X is C(O)NH.

14. The compound according to claim 1 wherein R⁹ is phenyl substituted with a carboxy, carboxy(C₁ to C₆ alkyl), tetrazolyl, tetrazolyl-N-(C₁ to C₆ alkyl)amino, carboxy(C₁ to C₆ alkyl)thio, carboxy(C₁ to C₆ alkyl)sulfonyl, (C₁ to C₆ alkyl)amino, or 5-oxo-2,5-dihydro[1,2,4]oxadiazolyl group; or R⁹ is a N-[carboxy(C₁ to C₆ alkyl)]indolinyl or N-[carboxy(C₁ to C₆ alkyl)]indolyl group.

15. The compound according to claim 14 wherein the phenyl group is substituted at its
3-position.

16. The compound according to claim 1 wherein R⁴ is
—(CH₂)_{q}-T-R¹⁰
wherein:
q is 0, 1, 2 or 3;
T is a bond, O, S, NH or N(C₁ to C₆ alkyl); and
R¹⁰ is C₁ to C₁₂ alkyl, C₃ to C₁₂ cycloalkyl, phenyl, naphthyl, pyridyl, pyrrolyl, imidazolyl, pyrazolyl, pyridazinyl, pyrimidinyl, triazolyl, furanyl, thienyl, furazanyl, oxazolyl, isoxazolyl, thiazolyl, thiazinyl, indolyl, indolinyl, isoindolyl, isoindolinyl, isoquinolinyl, quinolinyl, benzofuranyl, benzothienyl, piperazinyl, piperidinyl, pyrrolidinyl, pyrrolinyl, dihydropyranyl, tetrahydropyranyl, pyranyl, tetrahydrofuranyl, morpholinyl, thiazolidinyl, thiomorpholinyl or thioxanyl (all optionally substituted with 1, 2 or 3 groups independently selected from C₁ to C₆ alkyl, (C₁ to C₆ alkyl)oxy, C₃ to C₈ cycloalkyl, (C₃ to C₈ cycloalkyl)oxy, thio, (C₁ to C₆ alkyl)thio, carboxy, carboxy(C₁ to C₆ alkyl), formyl, (C₁ to C₆ alkyl)carbonyl, (C₁ to C₆ alkyl)oxycarbonyl, (C₁ to C₆ alkyl)carbonyloxy, nitro, trihalomethyl, hydroxy, hydroxy(C₁ to C₆ alkyl), amino, (C₁ to C₆ alkyl)amino, di(C₁ to C₆ alkyl)amino, aminocarbonyl, halo, halo(C₁ to C₆ alkyl), aminosulfonyl, (C₁ to C₆ alkyl)sulfonylamino or cyano).

17. The compound according to claim 16 wherein q is 0, T is a bond and R¹⁰ is C₁ to C₁₂ alkyl, C₃ to C₁₂ cycloalkyl, pyridyl or phenyl (all optionally substituted with OMe, NMe₂, CF₃, Me, F, Cl, Br or I).

18. The compound of formula (I) according to claim 17 wherein R⁴ is C₃₋₁₂ cycloalkyl.

19. The compound according to claim 1 wherein R⁴ is cyclohexyl.

20. A compound which is degraded in vivo to yield a compound according to claim 1.

21. A method of treating a gastrin related disorder comprising administering a therapeutically effective amount of a compound according to claim 1 to a patient in need thereof.

22. A method according to claim 21 wherein the gastrin related disorder is a gastrointestinal disorder or cancer.

23. A pharmaceutical composition comprising a compound of formula (I) according to claim 1 together with a pharmaceutically acceptable diluent or carrier.

24. (canceled)

25. (canceled)

26. The method of making a pharmaceutical composition according to claim 23 comprising mixing a compound of formula (I) with a pharmaceutically acceptable diluent or carrier.

27. A pharmaceutical composition comprising a proton pump inhibitor and a compound according to claim 1, together with a pharmaceutically acceptable diluent or carrier.

28. The composition according to claim 27 wherein the proton pump inhibitor is selected from (RS)-rabeprazole, (RS)-rabeprazole, lansoprazole, pantoprazole, (R)-omeprazole, (S)-omeprazole, perprazole, (R)-rabeprazole, (S)-rabeprazole, or the alkaline salts thereof.

29. A composition according to claim 27 wherein the proton pump inhibitor and the compound of formula (I) are each in an amount producing a therapeutically beneficial effect in patients suffering from gastrointestinal disorders.

30. A composition according to claim 29 wherein said therapeutically beneficial effect is a synergistic effect on the reduction of acid secretion in patients suffering from gastrointestinal disorders, or the prevention of gastrointestinal disorders in said patients, or the reduction of adverse effects associated with the one of the active ingredients by the other active ingredients.

31. A composition according to claim 27 wherein the amount of each of the active ingredients is equal to or less than that which is approved or indicated in monotherapy with said active ingredient.

32. A kit containing as a first active ingredient a compound according to claim 1, and as a second active ingredient a proton pump inhibitor, as a combined preparation for simultaneous, separate or sequential use in the treatment of patients suffering from gastrointestinal disorders.

33-38. (canceled)

39. A method of making a pharmaceutical composition comprising mixing a compound according to claim 1, and a proton pump inhibitor with a pharmaceutically acceptable diluent or carrier.

40. A method of treating a gastrointestinal disorder, comprising the simultaneous or sequential administration of a proton pump inhibitor and a compound according to claim 1 to a subject suffering from said disorder.

41. A method of reducing one or more adverse effects associated with administration of proton pump inhibitors in a patient suffering from a gastrointestinal disorder, comprising administering a compound according to claim 1 to a subject suffering from said disorder.

42. The method according to claim 41, wherein said compound is administered simultaneously or sequentially with said proton pump inhibitor.

43. The method according to claim 41 wherein said adverse effect is hyperplasia.
